SEQUENCE LISTING (16 sequences)

SEQ ID NO: 1 - DNA, 1704 bp, Anabaena variabilis
  CDS (1)..(1704)
atgaagacactatctcaagcacaaagcaaaacctcatctcaacaattttcttttactggaaattcttctgccaatgtaattattggtaatcagaaactcacaatcaatgatgttgcaagggtagcgcgtaatggcaccttagtgtctttaaccaataacactgatattttgcagggtattcaggcatcttgtgattacattaataatgctgttgaatctggggaaccaatttatggagtgacatctggttttggcggtatggccaatgttgccatatcccgtgaacaagcatctgaactccaaaccaacttagtttggttcctgaaaacaggtgcagggaacaaattacccttggcggatgtgcgcgcagctatgctcttgcgtgcaaactctcatatgcgcggtgcatctggcatcagattagaacttatcaagcgtatggagattttccttaacgctggtgtcacaccatatgtgtatgagtttggttcaattggtgcaagtggtgatttagtgccactatcctacattactggttcactgataggcttagatcccagttttaaggttgacttcaacggtaaagaaatggatgcgccaacagctctacgtcaactgaatttgtcacccttgacattgttgccgaaggaaggcttggcgatgatgaacggcacttcagtcatgacaggtattgcagcaaactgcgtctacgatactcaaattttaactgcgatcgctatgggcgttcacgctctagatatccaagctttaaacggaaccaatcaatcattccatccatttatccataattccaaaccacatcctggtcaattatgggcagcagatcagatgatttctttgttagccaattcccagttagttcgtgatgagttagatggtaaacacgattatcgtgatcacgagttgattcaagatcgttactcactccgatgccttccccagtatttggggccaatcgttgatggaatttcccagattgccaaacaaattgaaatcgaaatcaactcagtcaccgataacccactaattgatgttgataaccaagctagctatcatggaggaaatttcctcggacagtacgtgggtatgggaatggatcacctgcgttactatattgggttattggctaaacacctagatgtgcagattgccctcctcgcctcaccagagtttagcaatggactaccaccatctttattaggcaaccgagaacgtaaagtcaatatgggactcaaaggtctgcaaatatgcggtaactcaattatgccactgttgaccttctatggaaattccatcgccgatcgctttcctacccatgcagaacaatttaatcagaacatcaacagtcaaggatacacttcagcgactctagcccgccgttctgtggatatcttccagaattatgtggcgatcgctctgatgtttggagtccaagctgttgacctccgcacatataaaaagactggtcattacgatgcacgcgcctgtctatcacctgcaactgagcgcttatattcagcagtccgccacgtagttggacaaaaaccaacttcagatcgcccatatatttggaatgataatgagcaaggactggatgagcatattgcccggatttctgctgatatcgctgctggtggtgtgattgtgcaagcagttcaagatatcttaccctgcttgcattaa

SEQ ID NO: 2 - protein, 567 aa, Anabaena variabilis
MKTLSQAQSKTSSQQFSFTGNSSANVIIGNQKLTINDVARVARNGTLVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLH

SEQ ID NO: 3 - DNA, 1704 bp, Artificial Sequence
  Codon-optimized AvPAL sequence
atgaaaaccctgagccaggcacagagcaaaaccagcagccagcagtttagctttaccggcaatagcagcgcaaatgtgattattggtaatcagaaactgaccatcaatgatgttgcacgtgttgcccgtaatggcaccctggttagcctgaccaataataccgatattctgcagggtattcaggccagctgtgattatatcaataatgcagttgaaagcggtgaaccgatttatggtgttaccagcggttttggtggtatggcaaatgttgcaattagccgtgaacaggcaagcgaactgcagaccaatctggtttggtttctgaaaaccggtgcaggtaataaactgccgctggcagatgttcgtgcagcaatgctgctgcgtgcaaatagccatatgcgtggtgcaagcggtattcgtctggaactgattaaacgcatggaaatctttctgaatgccggtgttaccccgtatgtttatgaatttggtagcattggtgccagcggtgatctggttccgctgagctatattaccggtagcctgattggcctggatccgagctttaaagttgattttaatggcaaagaaatggacgcaccgaccgcactgcgtcagctgaatctgagtccgctgacactgctgccgaaagaaggtctggcaatgatgaatggcaccagcgttatgaccggtattgcagcaaattgtgtttatgatacccagattctgaccgcaattgcaatgggtgttcatgcactggatattcaggcactgaatggtacaaatcagagctttcatccgtttatccataacagcaaaccgcatccgggtcagctgtgggcagcagatcagatgattagcctgctggccaatagccagctggttcgtgatgaactggatggtaaacatgattatcgtgatcatgaactgatccaggatcgttatagcctgcgttgtctgccgcagtatctgggtccgattgttgatggtattagccagattgccaagcagattgaaattgaaattaacagcgttaccgacaatccgctgattgatgttgataatcaggcaagctatcatggtggtaattttctgggtcagtatgttggtatgggtatggatcatctgcgctattatatcggtctgctggcaaaacatctggatgttcagattgcactgctggcatcaccggaatttagcaatggtctgcctccgagtctgctgggtaatcgtgaacgtaaagttaatatgggtctgaaaggtctgcagatttgcggtaatagcattatgccgctgctgaccttttatggtaatagtattgcagatcgttttccgacacatgccgaacagtttaaccagaatattaacagccagggttataccagcgcaaccctggcacgtcgtagcgttgatatttttcagaattatgttgccattgcgctgatgtttggtgttcaggcagttgatctgcgtacctacaaaaaaaccggtcattatgatgcacgtgcctgtctgtcaccggcaaccgaacgtctgtatagcgcagttcgtcatgttgttggtcagaaaccgacctcagatcgtccgtatatttggaatgataatgaacaaggcctggatgaacatattgcacgtattagtgcagatattgcagccggtggtgttattgttcaggccgttcaggacattctgccgtgtctgcattaa

SEQ ID NO: 4 - DNA, 2202 bp, Plagiochasma appendiculatum
  CDS (1)..(2202)
atggcggcgatggtgatggaagtcggtacacctgcactggaccaagtgccagcagtgaagcctctcgtcgatggggctggggactggactgtccagctgtgcctcagtccagtgccgaagtcgaatgtgaactccgaggacccattgcagtgggcaagagctgccaaaacaattcaaggcagtcactgggaggaagtgaagagcctggtgagcgagttcgagcagtcgaaggaggtggtgattcaaggagtgacactgacagttgcacaggttaccgctgtggctcgaaatcccaatgtgaccgtggtgctcgacgcagccactgccaaggagagggtggacaagagcaacgactgggtcttgcaccacatggtaaagggaactgacacttacggtgtcaccactggcttcggagccacttctcacaggcggacgaatcaaggagtggagctgcagcgagagctcatccgatttcttaatgcaggcgtgttgtctgacgatgccagcatcctacccgtgagcacgaccagagccgcgatgctggtcagaaccaacactctcatgcaggggtactccggcattcgatgggaaattttgcagaccatgcagaagcttctgaacaagcacattactcccagactccctctgcgcggaaccatcacagcctcgggggatcttgtgcccttgtcttacatcgccggactgctcaccggccgccccaactctagagcttgcacgcaggatggcaaggtgttgacctcctcggaagcactgaagcaagtaggactgctcaagccattcgagttgcagcccaaggaaggactggctatggtgaatggaaccgcggttggtgcagcgcaggcctcgatggcttgctacgatgccaacttgttggcggtgttcgcagagatttcgtccgcgctcttctgtgaagccatgcaggggaagccagaattcacagatcatttgacgcacaagctgaagcaccatccgggccagattgaagcagccgcggtgatggagtggatgctggacggaagcgcatacatgaaagcggcggccaaactgcacgagactgaccctctgaagaagcccaagcaggacaggtacgctctccgcacatcgccgcagtggttgggccctcaaattgaagtgatccgcgcggccactcactccatcgagagggagatcaactccgtgaacgacaacccgctcatcgatgtgtccagagacaaggctctgcatggaggaaatttccaaggaaccccaattggagtgtcgatggacaacatgcgcttggcgcttgctgccatagggaagctcatgttcgcacaaatgtcagaattggtgaatgatttctacaacagtggattgccgtcgaacttaacgggcggcccgaacccgagcctggactacgggttcaaaggagccgagatcgccatggcttcttactgctccgagattcagtaccttgctaatccggtgactactcacgtgcagagtgcggagcagcacaatcaggacgtgaattctctagggttgatttcggctcgcaagacggccgaagctattgaggtcctgaagctcatgtcttcaacattcatggttgcgctctgccaggctgttgatctgagacacctagaggaaaacatgctcgcaggcgtgaagcaggtggttcaacaagtcgcgaagaaaaccctctttgccgacgaggctggagtgcttcttccttctcgcttctgcgaaaaggatctgctcaaagttgtcgatgatacaccccccttcacatacgtggacgacgccaccagcaaatcgtaccctcttatgacgaaattgagggaggtgctcgtgaaccacgccatcgagaactcgaagctcgatgataagtgttcggttttccgcagaatatccgttttcgaggaggaattgaagactcagttggaagtcaccgtcccccagatcagaagccaatatgacagtggcgtcacgtccattgcgaataagatcaaggaatgccgcaccttcccgatttacaacttcgttcgatctttgggaacgaagcttctggtcggaacagagacgaggtctcctggagaggacatccagctcgtttacgaggccattaacgagggcaaatttgccagtcctttgctatcttgcttggatggttggcatggagctcccatcccaattgatgtgtga

SEQ ID NO: 5 - protein, 733 aa, Plagiochasma appendiculatum
MAAMVMEVGTPALDQVPAVKPLVDGAGDWTVQLCLSPVPKSNVNSEDPLQWARAAKTIQGSHWEEVKSLVSEFEQSKEVVIQGVTLTVAQVTAVARNPNVTVVLDAATAKERVDKSNDWVLHHMVKGTDTYGVTTGFGATSHRRTNQGVELQRELIRFLNAGVLSDDASILPVSTTRAAMLVRTNTLMQGYSGIRWEILQTMQKLLNKHITPRLPLRGTITASGDLVPLSYIAGLLTGRPNSRACTQDGKVLTSSEALKQVGLLKPFELQPKEGLAMVNGTAVGAAQASMACYDANLLAVFAEISSALFCEAMQGKPEFTDHLTHKLKHHPGQIEAAAVMEWMLDGSAYMKAAAKLHETDPLKKPKQDRYALRTSPQWLGPQIEVIRAATHSIEREINSVNDNPLIDVSRDKALHGGNFQGTPIGVSMDNMRLALAAIGKLMFAQMSELVNDFYNSGLPSNLTGGPNPSLDYGFKGAEIAMASYCSEIQYLANPVTTHVQSAEQHNQDVNSLGLISARKTAEAIEVLKLMSSTFMVALCQAVDLRHLEENMLAGVKQVVQQVAKKTLFADEAGVLLPSRFCEKDLLKVVDDTPPFTYVDDATSKSYPLMTKLREVLVNHAIENSKLDDKCSVFRRISVFEEELKTQLEVTVPQIRSQYDSGVTSIANKIKECRTFPIYNFVRSLGTKLLVGTETRSPGEDIQLVYEAINEGKFASPLLSCLDGWHGAPIPIDV

SEQ ID NO: 6 - DNA, 2203 bp, Artificial Sequence
  Codon-optimized PaPAL sequence
atggcagcaatggtaatggaagtaggtacaccagcattagatcaagtaccggcagttaaaccgctggttgatggtgccggtgattggaccgttcagctgtgtctgagtccggttccgaaaagcaatgttaatagcgaagatccgctgcagtgggcacgtgcagcaaaaaccattcagggtagccattgggaagaagttaaaagcctggttagcgaatttgaacagagcaaagaagttgttattcagggcgttaccctgaccgttgcacaggttaccgcagttgcacgtaatccgaatgttaccgttgttctggatgcagcaaccgcaaaagaacgtgttgataaaagtaatgattgggtgctgcatcatatggttaaaggcaccgatacctatggtgttaccaccggttttggtgcaaccagccatcgtcgtaccaatcagggtgttgaactgcagcgtgaactgattcgttttctgaatgccggtgttctgagtgatgatgcaagcattctgccggttagcacaacccgtgcagccatgctggttcgtacaaataccctgatgcagggttatagcggtattcgttgggaaattctgcagaccatgcagaaactgctgaacaaacatattacaccgcgtctgccgctgcgtggcaccattaccgcaagcggtgatctggttccgctgagctatattgcaggtctgctgaccggtcgtccgaatagccgtgcatgtacccaggatggtaaagttctgaccagcagcgaagcactgaaacaggttggcctgctgaaaccgtttgagctgcagccgaaagaaggtctggccatggttaatggtacagcagttggtgcagcacaggcaagcatggcatgttatgatgcaaatctgctggcagtttttgcagaaatttcaagcgcactgttttgtgaagcaatgcagggtaaaccggaatttaccgatcatctgacccataaactgaaacatcatccgggtcagattgaagcagcagcagttatggaatggatgctggatggtagcgcatatatgaaagcagccgcaaaactgcatgaaaccgatccgctgaaaaaaccgaaacaggatcgttatgcactgcgtaccagtccgcagtggctgggtccgcagatcgaagttattcgtgcagcgacccatagcattgaacgtgaaattaacagcgttaatgataacccgctgattgatgttagccgtgataaagcactgcatggtggtaattttcagggcaccccgattggtgttagcatggataatatgcgtctggcactggcagccattggtaaactgatgtttgcacagatgagcgaactggtgaatgatttctataatagcggtctgccgagtaatctgacaggcggtccgaatccgagcctggattatggttttaaaggtgccgaaattgcaatggcaagctattgtagcgaaattcagtatctggcaaatccggttaccacacatgttcagagcgcagaacagcataatcaggatgtgaatagcctgggtctgattagcgcacgtaaaaccgcagaagcaattgaagttctgaaactgatgagcagcacctttatggttgcactgtgtcaggcagttgatctgcgtcatctggaagaaaacatgctggcaggcgttaaacaggtggttcagcaggttgcaaaaaaaaccctgtttgcagatgaagcgggtgttctgctgccgagccgtttttgtgaaaaagatctgctgaaagtggttgatgatacccctccgtttacctatgttgatgatgcgaccagcaaaagctatccgctgatgaccaaactgcgtgaagtgctggttaatcatgcaatcgaaaatagcaaactggacgataaatgcagcgtgtttcgtcgtattagcgtgtttgaagaagaactgaaaacccagctggaagttaccgttccgcagattcgtagccagtatgatagcggtgtgaccagcattgccaataaaatcaaagaatgtcgcacctttccgatctataactttgttcgtagcctgggcacaaaactgctggttggtacggaaacccgtagtccgggtgaagatattcagctggtttatgaagcaatcaacgaaggtaaatttgcaagtccgctgctgagctgtctggatggttggcatggtgcaccgattccgattgatgtgtaaa

SEQ ID NO: 7 - DNA, 2124 bp, Arabidopsis thaliana
  CDS (1)..(2124)
atggagctatgcaatcaaaacaatcacatcaccgccgtctcgggcgatccgttgaactggaacgcgacggccgaagctttgaaagggagccacctggatgaggtgaaacgaatggtgaaagagtataggaaagaggcggtgaagttaggaggtgagactttgacgattggtcaagtagccgccgtggctagaggaggaggaggatctacggtggagctagcggaggaggctcgtgccggagtcaaggcgagtagcgaatgggtgatggagagcatgaaccgaggaacggacagttatggagttaccacagggtttggtgcaacttcccatagaagaaccaaacaaggcggtgcacttcaaaatgagcttattaggttcttgaatgccggaatatttggccccggcgccggggacacgtcacacacgttgccaaagccgacaacaagagcggcaatgctcgtccgtgtcaacactctcctccaaggctactccggtatacgcttcgagattctcgaagcaattacaaagcttctcaaccacgaaatcactccgtgcctccctctccgtggcaccatcaccgcctccggtgaccttgttcctctctcttacatcgccggacttctcactggccgtcccaactccaaagccgtgggtccctctggtgagactctcactgcctctgaggcctttaagctcgccggagtatcgtcctttttcgagctgcagcctaaggaaggactagcacttgtgaacgggacagcggttggatcgggtttggcctcaacggttttgttcgatgcaaatattttggctgttttatcggaagttatgtctgccatgttcgcagaggttatgcaagggaaaccggagtttacagatcatcttacgcataagctcaagcaccatcccggtcagatcgaagccgccgcaattatggaacatatattagacggaagctcttacgttaaagaagctcaacttctccacgaaatggatcctcttcaaaaacctaaacaagatcggtacgctttacgtacgtcaccacaatggcttgggccgcagattgaagtgatcagagcggctactaaaatgattgagcgtgagatcaactctgttaatgataaccctttgatagatgtgtcgaggaacaaggcgttgcacggtggaaatttccaagggacaccgatcggtgttgccatggataattcccgtctagccattgcttccattgggaaactcatgtttgcgcagttttctgaactagtgaacgatttctacaacaatggtttgccttctaatctatctggtgggagaaaccctagtcttgattacgggtttaaaggcgcggaaatagccatggcttcttattgctccgagcttcagttcctggctaatcccgtgaccaaccatgtccaaagcgcagagcagcataaccaagacgttaattccctagggctaatctctagcaggaaaactgcagaagcagtggatatcctcaagctaatgtccacaacctacttagtcgcgctttgccaagccgttgatctaagacatcttgaagagaatctgaagaaggcggttaaatcagcagtgagtcaggtggcgaaacgggtcttaaccgttggtgccaacggggagctacatccgtcaaggttcacagaacgtgatgtcctccaagtggttgaccgagagtacgtgttctcatacgcagacgatccctgcagcctcacttacccgctaatgcagaaacttagacacattcttgtagaccacgctttagcggatccagaacgcgaggccaattccgcgacatcggttttccacaaaatcggagcttttgaagccgagctgaaactgcttctccctaaagaagtagaacgcgtccgggttgaatacgaggaaggaacatcggctatagctaaccggattaaggaatgtcggtcttatccattgtatcggtttgtccgcgatgagctaaatactgaactgcttactggagagaatgttcggtcgccaggagaggagtttgataaagtgttcttagcgatttctgatggaaaacttattgatccgttgttggaatgtctcaaggagtggaacggagctccggtttcaatctgttga

SEQ ID NO: 8 - protein, 707 aa, Arabidopsis thaliana
MELCNQNNHITAVSGDPLNWNATAEALKGSHLDEVKRMVKEYRKEAVKLGGETLTIGQVAAVARGGGGSTVELAEEARAGVKASSEWVMESMNRGTDSYGVTTGFGATSHRRTKQGGALQNELIRFLNAGIFGPGAGDTSHTLPKPTTRAAMLVRVNTLLQGYSGIRFEILEAITKLLNHEITPCLPLRGTITASGDLVPLSYIAGLLTGRPNSKAVGPSGETLTASEAFKLAGVSSFFELQPKEGLALVNGTAVGSGLASTVLFDANILAVLSEVMSAMFAEVMQGKPEFTDHLTHKLKHHPGQIEAAAIMEHILDGSSYVKEAQLLHEMDPLQKPKQDRYALRTSPQWLGPQIEVIRAATKMIEREINSVNDNPLIDVSRNKALHGGNFQGTPIGVAMDNSRLAIASIGKLMFAQFSELVNDFYNNGLPSNLSGGRNPSLDYGFKGAEIAMASYCSELQFLANPVTNHVQSAEQHNQDVNSLGLISSRKTAEAVDILKLMSTTYLVALCQAVDLRHLEENLKKAVKSAVSQVAKRVLTVGANGELHPSRFTERDVLQVVDREYVFSYADDPCSLTYPLMQKLRHILVDHALADPEREANSATSVFHKIGAFEAELKLLLPKEVERVRVEYEEGTSAIANRIKECRSYPLYRFVRDELNTELLTGENVRSPGEEFDKVFLAISDGKLIDPLLECLKEWNGAPVSIC

SEQ ID NO: 9 - DNA, 2124 bp, Artificial Sequence
  Codon-optimized AtPAL sequence
atggaattatgtaatcaaaataatcatattacagcagtatcaggtgatccgctgaattggaatgcaaccgcagaagcactgaaaggtagccatctggatgaagttaaacgtatggtgaaagagtatcgtaaagaggccgttaaattaggcggtgaaaccctgaccattggccaggttgcagcagttgcacgcggtggtggtggtagcaccgttgaactggccgaagaagcacgcgcaggcgttaaagcaagcagcgaatgggttatggaaagcatgaatcgtggcaccgatagttatggtgttaccaccggttttggtgcaaccagccatcgtcgtaccaaacaaggtggtgcactgcagaatgaactgattcgttttctgaatgccggtatttttggtcctggtgccggtgataccagtcataccctgccgaaaccgaccacacgtgcagcaatgctggttcgtgttaataccctgctgcagggctatagcggtattcgttttgaaattctggaagccattaccaaactgctgaaccatgaaattaccccgtgtctgccgctgcgtggcaccattaccgcaagcggtgatctggttccgctgagctatattgcaggtctgctgaccggtcgtccgaatagcaaagcagttggtccgagtggcgaaacactgaccgccagcgaagcatttaaactggctggtgttagcagcttttttgaactgcagccgaaagaaggtctggcactggttaatggcaccgcagttggtagcggtctggcaagcaccgttctgtttgatgcaaatattctggcagttctgagcgaagttatgagcgcaatgtttgcagaagttatgcagggtaaaccggaatttaccgatcatctgacccataaactgaaacatcatccgggtcagattgaagcagcagcaattatggaacatattctggatggtagcagctatgttaaagaagcacagctgctgcacgaaatggatccgctgcagaaaccgaaacaggatcgttatgcactgcgtaccagtccgcagtggctgggtccgcagatcgaagttattcgtgccgcaaccaaaatgattgaacgtgaaattaacagcgtgaacgataacccgctgattgatgttagccgtaataaagcactgcatggtggtaattttcagggcaccccgattggtgttgcaatggataatagccgtctggccattgcaagcattggtaaactgatgtttgcacagtttagcgaactggtgaacgatttctataataacggtctgccgagcaatctgagcggtggtcgtaatccgagcctggattatggttttaaaggtgcagaaattgccatggccagctattgcagcgagctgcagtttctggcaaatccggttaccaatcatgttcagagcgcagaacagcataatcaggatgttaatagcctgggtctgattagcagccgtaaaacagcagaagccgttgatattctgaaactgatgagcaccacctatctggttgccctgtgtcaggcagttgatctgcgtcatctggaagaaaatctgaaaaaagcagttaaaagcgcagttagccaggtggcaaaacgtgttctgaccgttggtgcaaatggtgaactgcatccgtcacgttttaccgaacgtgatgttctgcaggttgttgatcgtgaatatgtttttagctatgccgatgatccgtgtagcctgacctatccgctgatgcaaaaactgcgtcatatcctggttgatcatgcactggcagatccggaacgtgaagcaaatagcgcaaccagcgtttttcataaaattggtgcatttgaagccgagctgaaactgctgctgcctaaagaagttgaacgggttcgcgttgaatatgaagaaggcaccagcgcaattgccaatcgtattaaagaatgtcgttcgtatccgctgtatcgttttgttcgtgatgaactgaatacagaactgctgacaggtgaaaatgttcgtagtccgggtgaagaatttgataaagtttttctggcgattagcgacggcaaactgattgatcctctgctggaatgtctgaaagaatggaatggtgcaccggttagcatttgctaa

SEQ ID NO: 10 - DNA, 813 bp, Artificial Sequence
  Codon-optimized PfBesC sequence
  CDS (1)..(813)
atgggtgttcgtccggttagctatctggaaaatcagcgttgtccgcgtggtagccgtaaacaggcaatgagcattacccaagaaacctttcagagcgaaaccgcatatgttgttcgtaatgaaggtgttgatctggaactgaaactgtttatggatgagcagatcgagtatatcctgaaacatcgtgcaaccgaacatccgtttctgaatgcatatgccgaacatggtctgcctccggaacagagccaggttctgtacctggaaaccctgcattatttcaaatatctgccgttttatgtgtgcggcattagcaccattacacgtgatgaagcagttctgcgtaccattgcatttaatgcccgtgatgaactgggtgaaacccatagccatagcgatctgtatcgtaaatttctgcatgataaaggcatcagcgaagaacaaatcgaagcctataaatgtctgccgagcacacaggcactgaatgatggtatttgtgcactgtatagcaaaccgcctctgcagaaagcattaggtggtctgtttgcagatgaagcaatgagcgcaagcatggttagcaaatataacgatggcctgattaaagaaggtgtgggcgaacgcggtcgttttttttggaccctgcatatggaagttgaagtgggtcatagcaatgccgtttttaacgtgatggaaaaacatctgcagacaccgcaagaacgtcgcctgtttgccgaaggtattgaacagtatctgcatctgatggaagtttattgggatggcattgaacgtaaactgaatgccggtggtcgtcagtaa

SEQ ID NO: 11 - protein, 270 aa, Artificial Sequence
  Synthetic Construct
MGVRPVSYLENQRCPRGSRKQAMSITQETFQSETAYVVRNEGVDLELKLFMDEQIEYILKHRATEHPFLNAYAEHGLPPEQSQVLYLETLHYFKYLPFYVCGISTITRDEAVLRTIAFNARDELGETHSHSDLYRKFLHDKGISEEQIEAYKCLPSTQALNDGICALYSKPPLQKALGGLFADEAMSASMVSKYNDGLIKEGVGERGRFFWTLHMEVEVGHSNAVFNVMEKHLQTPQERRLFAEGIEQYLHLMEVYWDGIERKLNAGGRQ

SEQ ID NO: 12 - DNA, 1512 bp, Saccharomyces cerevisiae
  CDS (1)..(1512): ScFDC
atgaggaagctaaatccagctttagaatttagagactttatccaggtcttaaaagatgaagatgacttaatcgaaattaccgaagagattgatccaaatctcgaagtaggtgcaattatgaggaaggcctatgaatcccacttaccagccccgttatttaaaaatctcaaaggtgcttcgaaggatcttttcagcattttaggttgcccagccggtttgagaagtaaggagaaaggagatcatggtagaattgcccatcatctggggctcgacccaaaaacaactatcaaggaaatcatagattatttgctggagtgtaaggagaaggaacctctccccccaatcactgttcctgtgtcatctgcaccttgtaaaacacatatactttctgaagaaaaaatacatctacaaagcctgccaacaccatatctacatgtttcagacggtggcaagtacttacaaacgtacggaatgtggattcttcaaactccagataaaaaatggactaattggtcaattgctagaggtatggttgtagatgacaagcatatcactggtctggtaattaaaccacaacatattagacaaattgctgactcttgggcagcaattggaaaagcaaatgaaattcctttcgcgttatgttttggcgttcccccagcagctattttagttagttccatgccaattcctgaaggtgtttctgaatcggattatgttggcgcaatcttgggtgagtcggttccagtagtaaaatgtgagaccaacgatttaatggttcctgcaacgagtgagatggtatttgagggtactttgtccttaacagatacacatctggaaggcccatttggtgagatgcatggatatgttttcaaaagccaaggtcatccttgtccattgtacactgtcaaggctatgagttacagagacaatgctattctacctgtttcgaaccccggtctttgtacggatgagacacataccttgattggttcactagtggctactgaggccaaggagctggctattgaatctggcttgccaattctggatgcctttatgccttatgaggctcaggctctttggcttatcttaaaggtggatttgaaagggctgcaagcattgaagacaacgcctgaagaattttgtaagaaggtaggtgatatttactttaggacaaaagttggttttatagtccatgaaataattttggtggcagatgatatcgacatatttaacttcaaagaagtcatctgggcctacgttacaagacatacacctgttgcagatcagatggcttttgatgatgtcacttcttttcctttggctccctttgtttcgcagtcatccagaagtaagactatgaaaggtggaaagtgcgttactaattgcatatttagacagcaatatgagcgcagttttgactacataacttgtaattttgaaaagggatatccaaaaggattagttgacaaagtaaatgaaaattggaaaaggtacggatataaataa

SEQ ID NO: 13 - protein, 503 aa, Saccharomyces cerevisiae
MRKLNPALEFRDFIQVLKDEDDLIEITEEIDPNLEVGAIMRKAYESHLPAPLFKNLKGASKDLFSILGCPAGLRSKEKGDHGRIAHHLGLDPKTTIKEIIDYLLECKEKEPLPPITVPVSSAPCKTHILSEEKIHLQSLPTPYLHVSDGGKYLQTYGMWILQTPDKKWTNWSIARGMVVDDKHITGLVIKPQHIRQIADSWAAIGKANEIPFALCFGVPPAAILVSSMPIPEGVSESDYVGAILGESVPVVKCETNDLMVPATSEMVFEGTLSLTDTHLEGPFGEMHGYVFKSQGHPCPLYTVKAMSYRDNAILPVSNPGLCTDETHTLIGSLVATEAKELAIESGLPILDAFMPYEAQALWLILKVDLKGLQALKTTPEEFCKKVGDIYFRTKVGFIVHEIILVADDIDIFNFKEVIWAYVTRHTPVADQMAFDDVTSFPLAPFVSQSSRSKTMKGGKCVTNCIFRQQYERSFDYITCNFEKGYPKGLVDKVNENWKRYGYK

SEQ ID NO: 14 - DNA, 1512 bp, Artificial Sequence
  Codon-optimized ScFDC sequence
atgcgtaaactgaatccggcactggaatttcgtgattttattcaggttctgaaagatgaggatgacctgattgaaatcaccgaagaaattgatccgaatctggaagttggtgccattatgcgtaaagcatatgaaagccatctgcctgcaccgctgtttaaaaacctgaaaggtgcaagcaaagacctgtttagcattctgggttgtccggcaggtctgcgtagcaaagaaaaaggtgatcatggtcgtattgcccatcatctgggtttagatccgaaaaccaccatcaaagagatcatcgattatctgctggaatgcaaagagaaagaaccgctgcctccgattaccgttccggttagcagcgcaccgtgtaaaacccatattctgagcgaagaaaaaatccatctgcagagcctgccgacaccgtatctgcatgtttcagatggtggtaaatatctgcagacctatggtatgtggattctgcagacaccggacaaaaaatggaccaattggagcattgcacgtggtatggttgttgatgataaacatattaccggtctggtgattaaaccgcagcatattcgtcagattgcagatagctgggcagcaattggtaaagccaatgaaattccgtttgcactgtgttttggtgttccgcctgcagcaattctggtgagcagcatgccgattccggaaggtgttagcgaaagcgattatgttggtgcgattctgggtgaaagcgttccggtggttaaatgtgaaaccaatgatctgatggttccggcaaccagcgaaatggtttttgaaggcaccctgagcctgaccgatacacatctggaaggtccgtttggtgaaatgcatggttatgtgtttaaaagccagggtcatccgtgtccgctgtataccgttaaagcaatgagctatcgtgataatgcaattctgccggtgagcaatccgggtctgtgtaccgatgaaacccatacactgattggtagcctggttgccaccgaagcaaaagaactggcaattgaaagcggtctgccgattctggatgcatttatgccgtatgaagcacaggcactgtggctgattctgaaagttgatctgaaaggactgcaggcactgaaaaccacaccggaagaattttgtaaaaaagtgggcgatatctacttccgtaccaaagttggttttatcgtgcatgaaattattctggtggccgatgatatcgacatcttcaactttaaagaagtgatttgggcatacgtgacccgtcatacaccggttgcagatcagatggcatttgatgatgttaccagctttccgctggcaccgtttgttagccagagcagccgtagcaaaacaatgaaaggtggcaaatgtgtgaccaactgtatttttcgtcagcagtatgaacgcagcttcgattatatcacctgtaactttgaaaagggctatccgaaaggtctggtggataaagttaatgaaaactggaaacgctacggctataaataa

SEQ ID NO: 15 - DNA, 570 bp, Escherichia coli
  CDS (1)..(570): Flavin prenyltransferase
atgaaacgactcattgtaggcatcagcggtgccagcggcgcgatttatggcgtgcgcttattacaggttctgcgcgatgtcacagatatcgaaacgcatctggtgatgagccaggcagcgcgccagaccttatccctcgaaacggatttttctctgcgcgaagtgcaggcattagccgatgtcacgcacgatgcgcgcgatattgccgccagcatctcttccggttctttccagacgctggggatggtgattttaccctgttcaatcaaaaccctttccggcattgtccatagctatactgatggcttactgacccgtgcggcagatgtggtgctgaaagagcgtcgcccgttggtgctctgcgtgcgtgaaacaccattgcacttaggccatctgcgtttaatgactcaggcggcagaaatcggtgcggtgattatgcctcccgttccggcgttttatcatcgcccgcaatcccttgatgatgtgataaatcagacggttaatcgtgttcttgaccagtttgcgataacccttcctgaagatctctttgcccgctggcagggcgcataa

SEQ ID NO: 16 - protein, 189 aa, Escherichia coli
MKRLIVGISGASGAIYGVRLLQVLRDVTDIETHLVMSQAARQTLSLETDFSLREVQALADVTHDARDIAASISSGSFQTLGMVILPCSIKTLSGIVHSYTDGLLTRAADVVLKERRPLVLCVRETPLHLGHLRLMTQAAEIGAVIMPPVPAFYHRPQSLDDVINQTVNRVLDQFAITLPEDLFARWQGA

(19) United States
(12) Patent Application Publication
(10) Pub. No.: US 2025/0320481 A1
(43) Pub. Date: Oct. 16, 2025

(54) METHOD FOR PRODUCING CHAIN UNSATURATED CARBOXYLIC ACID COMPOUND USING PHENYLALANINE AMMONIA LAYSE

(71) Applicants: RIKEN, Wako-shi, Saitama (JP); ZEON CORPORATION, Tokyo (JP); THE YOKOHAMA RUBBER CO., LTD., Hiratsuka-shi, Kanagawa (JP)

(72) Inventors: Tomokazu SHIRAI, Wako-shi (JP); Yutaro MORI, Wako-shi (JP); Yoshihide YACHI, Tokyo (JP); Misao HIZA, Hiratsuka-shi (JP); Yayoi AKAHORI, Hiratsuka-shi (JP)

(73) Assignees: RIKEN, Wako-shi, Saitama (JP); ZEON CORPORATION, Tokyo (JP); THE YOKOHAMA RUBBER CO., LTD., Hiratsuka-shi, Kanagawa (JP)

(21) Appl. No.: 18/269,745

(22) PCT Filed: Dec. 7, 2021
(86) PCT No.: PCT/JP2021/044890

§ 371 Date: Jun. 26, 2023

(30) Foreign Application Priority Data
Dec. 28, 2020 (JP) 2020-219017

(51) Int. Cl. C12N 9/88; C12N 15/70; C12P 5/00; C12P 5/02; C12P 7/40; C12R 1/19

(52) U.S. Cl. CPC C12N 9/88; C12N 15/70; C12P 5/007; C12P 5/026; C12P 7/40; C12R 2001/19; C12Y 401/01; C12Y 403/01024

(57) ABSTRACT

A method for producing a chain-shaped unsaturated carboxylic acid compound or a geometric isomer thereof, having at least two carbon-carbon double bonds, including the steps of: removing, from a chain-shaped unsaturated carboxylic acid compound or a geometric isomer thereof having an amino group and a carbon-carbon double bond at a terminus, that amino group, in the presence of phenylalanine ammonia lyase, and further forming a carbon-carbon double bond.

### SEQUENCE LISTING

The present application contains a Sequence Listing that has been submitted electronically in ASCII format and is hereby incorporated by reference in its entirety. The ASCII copy, created on Jan. 24, 2024, is named Substitute_Sequence_Listing_ST25.txt and is 80,772 bytes in size.

### TECHNICAL FIELD

The present invention relates to a method for producing a third chain-shaped unsaturated carboxylic acid compound by introducing a further carbon-carbon double bond into a second chain-shaped unsaturated carboxylic acid compound having a carbon-carbon double bond at the terminus, in the presence of phenylalanine ammonia lyase. In addition, the present invention relates to a method for producing a second chain-shaped unsaturated carboxylic acid compound from a first chain-shaped unsaturated carboxylic acid compound having an amino group at the terminus, in the presence of terminal alkene-producing enzyme BesC, and then, from this compound, producing a third chain-shaped unsaturated carboxylic acid compound in the presence of phenylalanine ammonia lyase. Furthermore, the present invention relates to a method for producing such a third chain-shaped unsaturated carboxylic acid compound and then, in the presence of ferulic acid decarboxylase, producing a chain-shaped unsaturated hydrocarbon compound having carbon-carbon double bonds at both terminuses from the unsaturated carboxylic acid compound.

The present invention also relates to a phenylalanine ammonia lyase variant that can be used in these production methods, a DNA encoding the variant, a vector into which the DNA has been inserted, and a host cell into which the DNA or vector has been introduced. Furthermore, the present invention relates to a method for producing the variant using the aforementioned host cell.

### BACKGROUND ART

It can be said that butadiene (1,3-butadiene) is an extremely important organic compound in the chemical industry because it is used as a raw material for various polymer compounds, including various synthetic rubbers (such as butadiene rubber, styrene-butadiene rubber, and acrylonitrile-butadiene rubber), and polymer resins (such as ABS resin and Nylon 66). In addition, these polymer compounds using butadiene as a raw material are widely used not only for industrial products such as automobile tires but also for daily necessities such as clothing. Therefore, the demand for butadiene is increasing year by year. Its annual demand is 13 million tons, and the market size has reached 15 billion dollars.

Conventionally, butadiene has been produced by purifying the C4 fraction produced as a byproduct mainly in the process of producing ethylene and propylene from petroleum. However, due to environmental problems including the depletion of fossil fuels such as petroleum and global warming due to greenhouse gas emissions, there is an increasing need to achieve sustainable butadiene production to meet the above-mentioned increasing demand for butadiene. As a countermeasure against these problems, development has been actively conducted of a method which uses an enzyme to produce butadiene from a biomass resource-derived material that is a renewable resource.

For example, PTL 1 discloses a method for producing butadiene by using xylose as a raw material and a microorganism having an enzyme activity capable of converting xylose into crotyl alcohol or the like. In addition, PTL 2 discloses a method for producing butadiene by using xylose as a raw material and a microorganism having an enzyme activity capable of converting xylose into 2,3-butanediol. Many attempts have been made to produce unsaturated hydrocarbon compounds such as butadiene using an enzyme as described above.

Furthermore, it has been revealed by the present inventors that the production of 4-vinylguaiacol (4VG) by the decarboxylation reaction of ferulic acid involving ferulic acid decarboxylase (FDC) (see NPL 1) can be applied to the production of unsaturated hydrocarbon compounds such as butadiene (PTL 3). That is, it has been found by the present inventors that butadiene and the like can be produced through the decarboxylation reaction as shown in the following formula, using FDC, from muconic acid and the like.

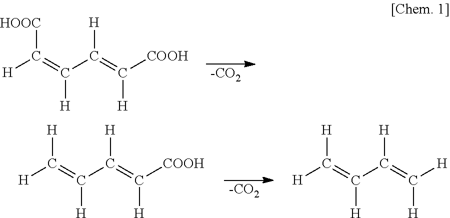

However, due to the high cost of muconic acid, there are concerns that it could hinder practical application. Therefore, it is desirable to develop a method for producing unsaturated compounds, such as butadiene, which have at least two carbon-carbon double bonds, more inexpensively using enzymes.

### CITATION LIST

### Patent Literature

[PTL 1] Japanese Patent Application Publication No. 2014-30376
[PTL 2] Japanese Patent Application Publication No. 2015-228804
[PTL 3] International Publication No. WO2019-022083

### Non Patent Literature

[NPL 1] Karl A. P. Payne et al., Nature. 2015, volume 522, issue 7557, pp. 497 to 501
[NPL 2]J A Marchand et al., Nature., 2019, volume 567, issue 7748, pp. 420 to 424

### SUMMARY OF INVENTION

### Technical Problem

The present invention has been made in view of the above-described problems of the related art, and an object thereof is to provide a method for producing an unsaturated compound having at least two carbon-carbon double bonds using an enzyme.

### Solution to Problem

The present inventors, in an effort to achieve the aforementioned objects, conducted intensive research and subsequently conceived the following reaction scheme to produce butadiene using L-lysine instead of muconic acid as the starting material. Note that L-lysine can be obtained relatively inexpensively, which can help control costs in the production of butadiene.

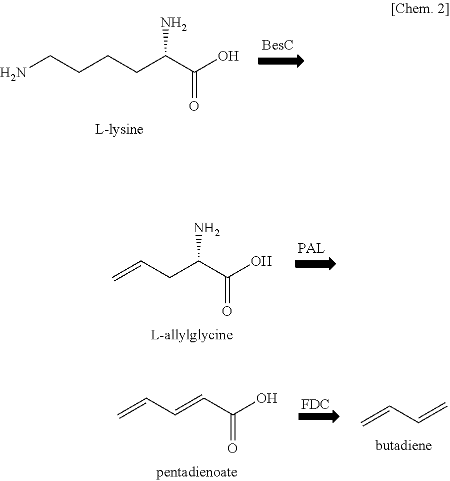

Furthermore, the present inventors conceived that the enzyme BesC disclosed in NPL 2 could be used in the production of L-allylglycine from L-lysine. This enzyme has an activity to catalyze a reaction that forms a carbon-carbon double bond at the terminus by oxidizing the propylamino group at the terminus to cleave the carbon-carbon bond within this group. In addition, they conceived that FDC, which has been revealed by the present inventors as described above, could be used in the production of butadiene from pentadienoate (PTL 3).

However, no reports of enzymes that could contribute to the production of pentadienoate from L-allylglycine were found. Therefore, the present inventors considered whether phenylalanine ammonia lyase (PAL), which is involved in the production of cinnamic acid from phenylalanine, could be utilized for this purpose.

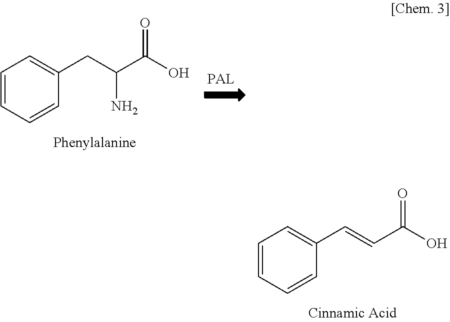 [SMILES: NC(Cc1ccccc1)C(=O)O ; O=C(O)/C=C/c1ccccc1]

Then, in practice, E. coli that highly expressed BesC and PAL derived from Arabidopsis thaliana (AtPAL) were cultivated. The resultant cell lysate obtained through processing was mixed with the cell lysate obtained from the cultivation and processing of E. coli that highly expressed FDC. Upon the addition of L-lysine, it was clearly revealed that butadiene was produced.

Furthermore, the present inventors verified butadiene production in the same manner for PALs derived from different sources (PAL from Anabaena variabilis (AvPAL), and PAL from Plagiochasma appendiculatum (PaPAL)). As a result, it was revealed that butadiene could be produced in the same way as with AtPAL using either AvPAL or PaPAL. Moreover, among these, the present inventors found that AvPAL had outstanding catalytic activity related to the production of pentadienoate.

Furthermore, regarding this AvPAL, the present inventors introduced a variety of amino acid substitutions and verified their impact on butadiene production. As a result, they found that the butadiene production quantity improved in comparison to the wild type prior to introduction in any of the following amino acid substitutions (1) to (5), leading to the completion of the present invention:
(1) substitution of leucine at position 108 with methionine, phenylalanine, or valine,
(2) substitution of phenylalanine at position 107 with tryptophan,
(3) substitution of leucine at position 219 with isoleucine,
(4) substitution of asparagine at position 223 with isoleucine,
(5) substitution of leucine at position 104 with alanine.

Specifically, the present invention relates to a method for producing a third chain-shaped unsaturated carboxylic acid compound by introducing a further carbon-carbon double bond into a second chain-shaped unsaturated carboxylic acid compound having a carbon-carbon double bond at the terminus, in the presence of PAL. In addition, the present invention relates to a method for producing a second chain-shaped unsaturated carboxylic acid compound from a first chain-shaped unsaturated carboxylic acid compound having an amino group at the terminus, in the presence of BesC, and then, from this compound, producing a third chain-shaped unsaturated carboxylic acid compound in the presence of phenylalanine ammonia lyase. Furthermore, the present invention relates to a method for producing such a third chain-shaped unsaturated carboxylic acid compound and, in the presence of PDC, producing a chain-shaped unsaturated hydrocarbon compound having carbon-carbon double bonds at both terminuses from the unsaturated carboxylic acid compound.

The present invention also relates to a PAL mutant that can be used in these production methods, a DNA encoding the mutant, a vector into which the DNA has been inserted, and a host cell into which the DNA or vector has been introduced. Furthermore, the present invention relates to a method for producing the mutant using the aforementioned host cell.

More specifically, the present invention provides the following.

[1]A method for producing a chain-shaped unsaturated carboxylic acid compound or a geometric isomer thereof, represented by the following formula (3), including the steps of: removing a first amino group from a chain-shaped unsaturated carboxylic acid compound or a geometric isomer thereof, having a first amino group and a first carbon-carbon double bond at a terminus, represented by the following formula (2), in the presence of phenylalanine ammonia lyase, and forming a second carbon-carbon double bond

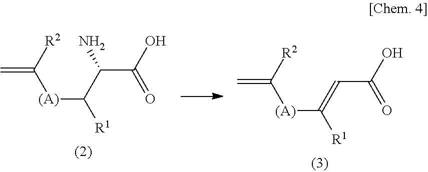

[in the formulas, (A) represents a linear hydrocarbon group having 0 to 5 carbon atoms, which may be substituted, and when the number of carbon atoms is 2 to 5, it may form a double bond between adjacent carbon atoms. R¹ and R² each independently represent a hydrogen atom, a linear or branched alkyl group having 1 to 5 carbon atoms, a linear or branched alkoxy group having 1 to 5 carbon atoms, or a hydroxy group.].
[2] The method according to [1], further including the steps of: removing a second amino group and a methylene group from a chain-shaped carboxylic acid compound or a geometric isomer thereof, having a first amino group and a second amino group at a terminus, represented by the following formula (1), in the presence of the terminal alkene generating enzyme BesC, and producing a chain-shaped unsaturated carboxylic acid compound or a geometric isomer thereof, represented by the formula (2), wherein the compound or a geometric isomer thereof is used to produce the chain-shaped unsaturated carboxylic acid compound or a geometric isomer thereof, represented by the formula (3)

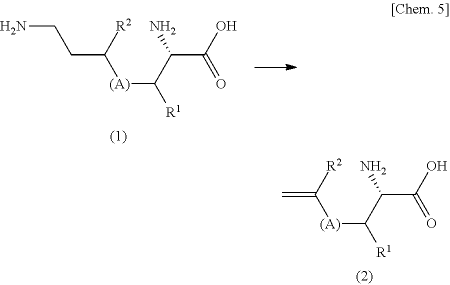

[in the formulas, (A) represents a linear hydrocarbon group having 0 to 5 carbon atoms, which may be substituted, and when the number of carbon atoms is 2 to 5, it may form a double bond between adjacent carbon atoms. R¹ and R² each independently represent a hydrogen atom, a linear or branched alkyl group having 1 to 5 carbon atoms, a linear or branched alkoxy group having 1 to 5 carbon atoms, or a hydroxy group.].
[3]A method for producing a chain-shaped unsaturated hydrocarbon compound or a geometric isomer thereof, represented by the following formula (4), further including the steps of: producing the chain-shaped unsaturated carboxylic acid compound or a geometric isomer thereof, represented by the formula (3) by the method according to [1] or [2], and decarboxylating a carboxyl group from the unsaturated carboxylic acid compound or a geometric isomer thereof, in the presence of ferulic acid decarboxylase

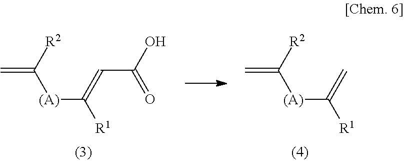

[in the formulas, (A) represents a linear hydrocarbon group having 0 to 5 carbon atoms, which may be substituted, and when the number of carbon atoms is 2 to 5, it may form a double bond between adjacent carbon atoms. R¹ and R² each independently represent a hydrogen atom, a linear or branched alkyl group having 1 to 5 carbon atoms, a linear or branched alkoxy group having 1 to 5 carbon atoms, or a hydroxy group.].
[4] The method according to any one of [1] to [3], where the phenylalanine ammonia lyase has at least one of the following features (1) to (5):
(1) position 108 in the amino acid sequence set forth in SEQ ID NO: 2, or an amino acid corresponding to the position, is methionine, phenylalanine, or valine,
(2) position 107 in the amino acid sequence set forth in SEQ ID NO: 2, or an amino acid corresponding to the position, is tryptophan,
(3) position 219 in the amino acid sequence set forth in SEQ ID NO: 2, or an amino acid corresponding to the position, is isoleucine,
(4) position 223 in the amino acid sequence set forth in SEQ ID NO: 2, or an amino acid corresponding to the position, is isoleucine,
(5) position 104 in the amino acid sequence set forth in SEQ ID NO: 2, or an amino acid corresponding to the position, is alanine.
   [5] A phenylalanine ammonia lyase variant into which at least one of the following amino acid substitutions (1) to (5) has been introduced:
(1) position 108 in the amino acid sequence set forth in SEQ ID NO: 2, or an amino acid corresponding to the position, is substituted with methionine, phenylalanine, or valine,
(2) position 107 in the amino acid sequence set forth in SEQ ID NO: 2, or an amino acid corresponding to the position, is substituted with tryptophan,
(3) position 219 in the amino acid sequence set forth in SEQ ID NO: 2, or an amino acid corresponding to the position, is substituted with isoleucine,
(4) position 223 in the amino acid sequence set forth in SEQ ID NO: 2, or an amino acid corresponding to the position, is substituted with isoleucine,
(5) position 104 in the amino acid sequence set forth in SEQ ID NO: 2, or an amino acid corresponding to the position, is substituted with alanine.
   [6]A DNA encoding the phenylalanine ammonia lyase variant according to [5].
   [7]A vector including the DNA according to [6].
   [8]A host cell into which the DNA according to [6] or the vector according to [7] has been introduced.
   [9]A method for producing a phenylalanine ammonia lyase variant, including: culturing the host cell according to [8] and collecting a protein expressed in the host cell.
   [10]A method for producing a phenylalanine ammonia lyase variant, including the step of: introducing at least one of the following amino acid substitutions (1) to (5) into a phenylalanine ammonia lyase:
(1) substituting position 108 in the amino acid sequence set forth in SEQ ID NO: 2, or an amino acid corresponding to the position, with methionine, phenylalanine, or valine,
(2) substituting position 107 in the amino acid sequence set forth in SEQ ID NO: 2, or an amino acid corresponding to the position, with tryptophan,
(3) substituting position 219 in the amino acid sequence set forth in SEQ ID NO: 2, or an amino acid corresponding to the position, with isoleucine,
(4) substituting position 223 in the amino acid sequence set forth in SEQ ID NO: 2, or an amino acid corresponding to the position, with isoleucine,
(5) substituting position 104 in the amino acid sequence set forth in SEQ ID NO: 2, or an amino acid corresponding to the position, with alanine.

### Advantageous Effects of Invention

The present invention makes it possible to provide a method for producing an unsaturated compound having at least two carbon-carbon double bonds using an enzyme. For example, the present invention also makes it possible to produce butadiene using relatively inexpensive L-lysine as a starting material.

### BRIEF DESCRIPTION OF DRAWINGS

FIG. 1 is a graph showing the results of measuring the amount of styrene produced by adding phenylalanine to a mixed solution of phenylalanine ammonia lyase derived from Arabidopsis thaliana, Anabaena variabilis, or Plagiochasma appendiculatum (AtPAL, AvPAL, or PaPAL), and ferulic acid decarboxylase.

FIG. 2 is a graph showing the results of measuring the amount of butadiene produced by adding allylglycine to a mixed solution of AtPAL, AvPAL, or PaPAL and ferulic acid decarboxylase.

FIG. 3 shows, with butadiene production quantity when using wild-type PAL set as 1.0, butadiene production quantity when using PAL variant.

### DESCRIPTION OF EMBODIMENTS

As demonstrated in the following examples, the present inventors have revealed that it is possible to produce unsaturated hydrocarbon compounds, which have carbon-carbon double bonds at both terminuses, such as butadiene, through a three-step reaction using three types of enzymes, as described below.

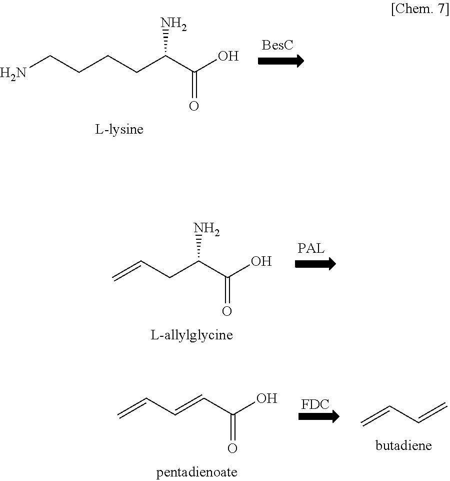

Specifically, the present inventors have revealed that phenylalanine ammonia lyase (PAL) can be used as an enzyme involved in the production of pentadienoate from L-allylglycine.

Therefore, the present invention relates to a method for producing a chain-shaped unsaturated compound having at least two carbon-carbon double bonds, which includes the following reaction step.

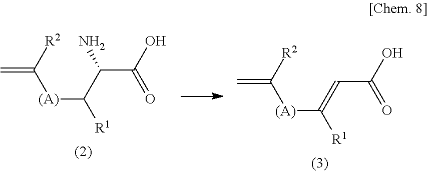

### <Method for Producing Third Chain-Shaped Unsaturated Carboxylic Acid Compound>

As described above, the present invention provides a method for producing a chain-shaped unsaturated carboxylic acid compound or a geometric isomer thereof, represented by the formula (3) (third chain-shaped unsaturated carboxylic acid compound), including the steps of: removing a first amino group from a chain-shaped unsaturated carboxylic acid compound or a geometric isomer thereof, having a first amino group and a first carbon-carbon double bond at a terminus, represented by the formula (2) (second chain-shaped unsaturated carboxylic acid compound), in the presence of phenylalanine ammonia lyase, and forming a second carbon-carbon double bond.

In the present invention, the “third chain-shaped unsaturated carboxylic acid compound” produced in the aforementioned reaction means a chain-shaped unsaturated carboxylic acid compound or a geometric isomer thereof, represented by the formula (3), which has at least two carbon-carbon double bonds, including a carbon-carbon double bond at the terminus.

In the present invention, (A) in each chemical formula represents a linear hydrocarbon group having 0 to 5 carbon atoms, which may be substituted. Note that the term “linear hydrocarbon group having 0 carbon atoms” implies that the carbon atoms bonded via (A) in compounds represented by each chemical formula, as well as geometric isomers thereof, are directly bonded without going through (A). Furthermore, when the number of carbon atoms in the substitutable linear hydrocarbon group is 2 to 5, at least one double bond may be formed between adjacent carbon atoms. In addition, substituents that the hydrocarbon group in (A) may have include, for example, linear or branched alkyl groups having 1 to 5 carbon atoms, linear or branched alkoxy groups having 1 to 5 carbon atoms, hydroxy groups, halogen atoms (such as fluorine, chlorine, bromine, and iodine), nitro groups, cyano groups, amino groups, carboxyl groups, and formyl groups. (A) in each chemical formula is preferably a linear hydrocarbon group having 0 carbon atoms.

In the present invention, R¹ and R² in each chemical formula each independently represent a hydrogen atom, a linear or branched alkyl group having 1 to 5 carbon atoms, a linear or branched alkoxy group having 1 to 5 carbon atoms, or hydroxy group. Examples of the “linear or branched alkyl group having 1 to 5 carbon atoms” include a methyl group, an ethyl group, an n-propyl group, an i-propyl group, an n-butyl group, an i-butyl group, an s-butyl group, a t-butyl group, an n-pentyl group, and an i-pentyl group. Examples of the “linear or branched alkoxy group having 1 to 5 carbon atoms” include a methoxy group, an ethoxy group, an n-propoxy group, an i-propoxy group, an n-butoxy group, an i-butoxy group, an s-butoxy group, a t-butoxy group, an n-pentyloxy group, an i-pentyloxy group, an n-pentyloxy group, and a 1,2-dimethyl-propoxy group. R¹ in each chemical formula is preferably a hydrogen atom. R² is preferably a hydrogen atom or a methyl group.

In each chemical formula, the combinations of (A), R¹, and R² are preferably each a linear hydrocarbon group having zero carbon atoms, a hydrogen group, and a hydrogen group, or a linear hydrocarbon group having zero carbon atoms, a hydrogen group, and a methyl group. Also, in the present invention, the “third chain-shaped unsaturated carboxylic acid compound” is preferably pentadienoate, 4-methylpentadienoate, or 3-methylpentadienoate.

It suffices that the condition of deaminating the second chain-shaped unsaturated carboxylic acid compound in the presence of phenylalanine ammonia lyase according to the present invention is a condition in which the deamination is promoted and a third chain-shaped unsaturated carboxylic acid compound is produced. Those skilled in the art can appropriately adjust and set the composition of the reaction liquid, the pH of the reaction liquid, the reaction temperature, the reaction time, and the like.

For example, the reaction liquid added with the phenylalanine ammonia lyase according to the present invention and its substrate, the second chain-shaped unsaturated carboxylic acid compound, is not particularly limited as long as it does not interfere with the reaction, but preferably a buffer solution having a pH of 6 to 8 and more preferably a buffer solution having a pH of 6 to 7 and containing potassium chloride and sodium phosphate.

In addition, the reaction temperature is not particularly limited either as long as the reaction is not hindered, but is usually 20 to 40° C. and preferably 25 to 37° C. Moreover, the reaction time is not particularly limited as long as it is a time for which the unsaturated hydrocarbon compound can be produced, but is usually 30 minutes to 7 days and preferably 12 hours to 2 days.

In addition, the third chain-shaped unsaturated carboxylic acid compound produced can be collected by appropriately using a known recovery and purification method (such as distillation and chromatography). Moreover, these methods may be carried out alone or may be carried out in multiple steps in appropriate combination.

### (Phenylalanine Ammonia Lyase)

The “phenylalanine ammonia lyase” is an enzyme registered as EC number: 4.3.1.24, and means an enzyme which catalyzes a reaction that uses phenylalanine as a substrate to produce cinnamic acid and ammonia. In addition, it is an enzyme also known as PAL, thylases, phenylalanine deaminase, tyrosine ammonia lyase, L-tyrosine ammonia lyase, phenylalanine ammonium lyase, and L-phenylalanine ammonia lyase.

As demonstrated in the following examples, it can be involved in the production of the third chain-shaped unsaturated carboxylic acid compound mentioned earlier, regardless of its origin. Therefore, there are no specific restrictions on the phenylalanine ammonia lyase according to the present invention, and various biologically derived ones can be used. For example, phenylalanine ammonia lyases can come from sources such as Anabaena variabilis, Arabidopsis thaliana, Plagiochasma appendiculatum, Rhodotorula glutinis, Planctomyces brasiliensis, Oryza sativa, Bambusa oldhamii, Taxus chinensis, Nicotiana tabacum, Streptomyces maritimus, Salvia miltiorrhiza, and Solanum lycopersicum. Among these, the phenylalanine ammonia lyase derived from Anabaena variabilis (phenylalanine ammonia lyase composed of the amino acid sequence set forth in SEQ ID NO: 2) is preferable from the viewpoint of having a higher catalytic activity to produce the third chain-shaped unsaturated carboxylic acid compound, as demonstrated in the following examples.

Additionally, the phenylalanine ammonia lyase according to the present invention has identity with the amino acid sequence set forth in SEQ ID NO: 2 by preferably 15% or more (for example, 16% or more, 17% or more, 18% or more, 19% or more), more preferably 20% or more (for example, 30% or more, 40% or more), further preferably 50% or more (for example, 60% or more, 70% or more), more preferably 80% or more (for example, 85% or more, 86% or more, 87% or more, 88% or more, 89% or more), and more preferably 90% or more (for example, 91% or more, 92% or more, 93% or more, 94% or more, 95% or more, 96% or more, 97% or more, 98% or more, 99% or more). Note that the term “identity” to the amino acid sequence set forth in SEQ ID NO: 2 means the proportion (%) of the number of amino acids that matches the amino acid sequence set forth in SEQ ID NO: 2 in the phenylalanine ammonia lyase according to the present invention, based on the total number of amino acids in the phenylalanine ammonia lyase according to the present invention.

Additionally, the phenylalanine ammonia lyase according to the present invention may be one in which mutations have been introduced naturally or unnaturally (artificially) into the amino acid sequence set forth in SEQ ID NO: 2. In other words, the phenylalanine ammonia lyase according to the present invention may also include proteins composed of amino acid sequences in which one or more amino acids in the amino acid sequence of phenylalanine ammonia lyase (such as the amino acid sequence set forth in SEQ ID NO: 2) have been substituted, deleted, added, and/or inserted. The term “more” here implies no particular restriction, but typically it is 2 to 200, preferably 2 to 150, more preferably 2 to 100, further preferably 2 to 70, more preferably 2 to 50, further preferably 2 to 30, more preferably 2 to 20, and further preferably 2 to 10 (for example, 2 to 9, 2 to 8, 2 to 7, 2 to 6, 2 to 5, 2 to 4, 2 or 3, or 2).

Furthermore, as demonstrated in the following examples, the phenylalanine ammonia lyase according to the present invention is preferably a phenylalanine ammonia lyase that has at least one of the following features (1) to (5):
(1) position 108 in the amino acid sequence set forth in SEQ ID NO: 2, or an amino acid corresponding to the position, is methionine, phenylalanine, or valine,
(2) position 107 in the amino acid sequence set forth in SEQ ID NO: 2, or an amino acid corresponding to the position, is tryptophan,
(3) position 219 in the amino acid sequence set forth in SEQ ID NO: 2, or an amino acid corresponding to the position, is isoleucine,
(4) position 223 in the amino acid sequence set forth in SEQ ID NO: 2, or an amino acid corresponding to the position, is isoleucine,
(5) position 104 in the amino acid sequence set forth in SEQ ID NO: 2, or an amino acid corresponding to the position, is alanine.

Note that in the present invention, regarding phenylalanine ammonia lyase, the term “corresponding positions” refers to the positions that align with each of leucine at position 108, phenylalanine at position 107, leucine at position 104, leucine at position 219, and asparagine at position 223 in the amino acid sequence set forth in SEQ ID NO: 2. This alignment is achieved when comparing the amino acid sequence set forth in SEQ ID NO: 2 with the amino acid sequences of phenylalanine ammonia lyases and the like from other species, using nucleotide and amino acid sequence analysis software (such as GENETYX-MAC and Sequencher) and BLAST (http://blast.ncbi.nlm.nih.gov/Blast.cgi).

Moreover, in relation to the features (1) to (5), it is preferable to have a phenylalanine ammonia lyase that possesses at least one of the following features: position 108 in the amino acid sequence set forth in SEQ ID NO: 2, or an amino acid corresponding to the position, is methionine; position 107 in the amino acid sequence set forth in SEQ ID NO: 2, or an amino acid corresponding to the position, is tryptophan; and position 108 in the amino acid sequence set forth in SEQ ID NO: 2, or an amino acid corresponding to the position, is phenylalanine. It is more preferable to have a phenylalanine ammonia lyase that possesses at least one of the following features: position 108 in the amino acid sequence set forth in SEQ ID NO: 2, or an amino acid corresponding to the position, is methionine; and position 107 in the amino acid sequence set forth in SEQ ID NO: 2, or an amino acid corresponding to the position, is tryptophan.

Furthermore, the phenylalanine ammonia lyase, which has specific amino acids at such sites, may be a wild type phenylalanine ammonia lyase, and it may also be a mutant phenylalanine ammonia lyase into which amino acid substitution has been introduced so as to have at least one of the features (1) to (5).

Such mutants more specifically include any of the following (a) to (c).
(a) A phenylalanine ammonia lyase mutant including an amino acid sequence in which at least one of the following amino acid substitutions (1) to (5) has been introduced, in the amino acid sequence set forth in SEQ ID NO: 2
(1) position 108 in the amino acid sequence set forth in SEQ ID NO: 2, or an amino acid corresponding to the position, is substituted with methionine, phenylalanine, or valine,
(2) position 107 in the amino acid sequence set forth in SEQ ID NO: 2, or an amino acid corresponding to the position, is substituted with tryptophan,
(3) position 219 in the amino acid sequence set forth in SEQ ID NO: 2, or an amino acid corresponding to the position, is substituted with isoleucine,
(4) position 223 in the amino acid sequence set forth in SEQ ID NO: 2, or an amino acid corresponding to the position, is substituted with isoleucine,
(5) position 104 in the amino acid sequence set forth in SEQ ID NO: 2, or an amino acid corresponding to the position, is substituted with alanine.
(b) A phenylalanine ammonia lyase mutant including an amino acid sequence, in the amino acid sequence set forth in SEQ ID NO: 2, where at least one of the amino acid substitutions (1) to (5) has been introduced, and furthermore, one or more amino acids are substituted, deleted, added, and/or inserted at one or several positions other than the aforementioned positions.
(c) A phenylalanine ammonia lyase mutant including an amino acid sequence, which has at least 15% identity with the amino acid sequence set forth in SEQ ID NO: 2, the amino acid sequence having at least one of the following amino acid substitutions (1) to (5) introduced thereto.

The “identity” with the amino acid sequence set forth in SEQ ID NO: 2, of the “more” amino acids that have been substituted, deleted, added, and/or inserted, is as previously described, including their suitable forms (ranges). Additionally, mutants of phenylalanine ammonia lyase may occur naturally or unnaturally (artificially).

In other words, this includes phenylalanine ammonia lyase variants where amino acid substitutions and the like have been artificially introduced.

Note that whether phenylalanine ammonia lyase, or natural or non-natural mutants thereof, possess catalytic activity to produce a third chain-shaped unsaturated carboxylic acid compound can be determined by those skilled in the art through known methods (such as chromatography mass spectrometry) by directly measuring the quantity of the unsaturated carboxylic acid compound that is produced.

In addition, the phenylalanine ammonia lyase according to the present invention may be added with additional compounds directly or indirectly. Such addition is not particularly limited, and may be addition at the gene level or chemical addition. Further, the addition position is not particularly limited either, and may be one of the amino terminus and the carboxyl terminus of the phenylalanine ammonia lyase according to the present invention, or may be both of them. The addition at the gene level can be achieved by using a DNA encoding the phenylalanine ammonia lyase according to the present invention, the DNA added with a DNA encoding a different protein with matched reading frames. There is no particular limitation on the “different protein” thus added. For the purpose of facilitating the purification of the phenylalanine ammonia lyase according to the present invention, a tag protein for purification such as polyhistidine (His-) tag protein, FLAG-tag protein (registered trademark, Sigma-Aldrich), or glutathione-S-transferase (GST) is preferably used. In addition, for the purpose of facilitating the detection of the phenylalanine ammonia lyase according to the present invention, a tag protein for detection including a fluorescent protein such as GFP and a chemiluminescent protein such as luciferase is preferably used. The chemical addition may be covalent bond or non-covalent bond. The “covalent bond” is not particularly limited, and examples thereof include an amide bond between an amino group and a carboxyl group, an alkylamine bond between an amino group and an alkyl halide group, a disulfide bond between thiols, and a thioether bond between a thiol group and a maleimide group or an alkyl halide group. Examples of the “non-covalent bond” include a biotin-avidin bond. In addition, as the “additional compounds” thus chemically added, for example, fluorescent dyes such as Cy3 and rhodamine are preferably used for the purpose of facilitating the detection of the phenylalanine ammonia lyase according to the present invention.

In addition, the phenylalanine ammonia lyase according to the present invention may be used by being mixed with additional components. The additional components are not particularly limited, and examples thereof include sterilized water, physiological saline, vegetable oil, surfactants, lipids, solubilizers, buffer agents, protease inhibitors, and preservatives.

### <Method for Producing Third Chain-Shaped Unsaturated Carboxylic Acid Compound Through Production of Second Chain-Shaped Unsaturated Carboxylic Acid Compound>

As demonstrated in the following examples, the present inventors have found that the second chain-shaped unsaturated carboxylic acid compound, which serves as a raw material for the production of the aforementioned third chain-shaped unsaturated carboxylic acid compound, can also be produced as shown in the reaction below.

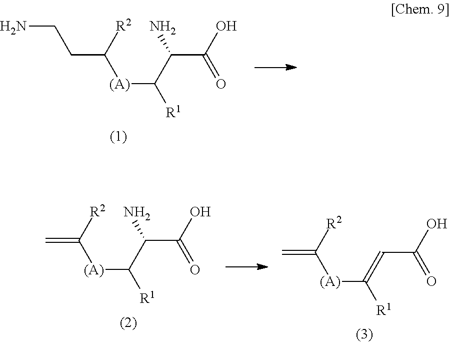

That is, the present inventors have found that it is possible to produce a second chain-shaped unsaturated carboxylic acid compound, represented by the formula (2), by oxidizing the propylamino group at the terminus of the chain-shaped unsaturated carboxylic acid compound or a geometric isomer thereof (the first chain-shaped unsaturated carboxylic acid compound) represented by the formula (1) using the enzyme BesC, thereby cleaving the carbon-carbon bond in the group and forming a carbon-carbon double bond at the terminus. Therefore, the present invention can also take the form of a method for producing a third chain-shaped unsaturated carboxylic acid compound through the production of the second chain-shaped unsaturated carboxylic acid compound.

In the present invention, the “second chain-shaped unsaturated carboxylic acid compound” means a chain-shaped unsaturated carboxylic acid compound or a geometric isomer thereof, as represented by the formula (2), which possesses a first amino group and a first carbon-carbon double bond at the terminus. Furthermore, the “first chain-shaped unsaturated carboxylic acid compound”, which serves as a raw material for its production, means a chain-shaped unsaturated carboxylic acid compound or a geometric isomer thereof, as represented by the formula (1), possessing a first amino group and a second amino group at the terminus.

In these compounds, for (A) and R¹, including their preferable embodiments, they are as mentioned above, and the “second chain-shaped unsaturated carboxylic acid compound” is preferably L-allylglycine, L-(2-methylallyl)glycine, or L-(3-methylallyl)glycine. The “first chain-shaped unsaturated carboxylic acid compound” is preferably L-lysine, 4-methyllysine, or 3-methyllysine.

It suffices that the condition of forming a carbon-carbon double bond at the terminus of the first chain-shaped unsaturated carboxylic acid compound in the presence of the BesC according to the present invention is a condition in which the formation of the double bond is promoted and a second chain-shaped unsaturated carboxylic acid compound is produced. Those skilled in the art can appropriately adjust and set the composition of the reaction liquid, the pH of the reaction liquid, the reaction temperature, the reaction time, and the like.

For example, the reaction liquid added with the BesC according to the present invention and its substrate, the first chain-shaped unsaturated carboxylic acid compound, is not particularly limited as long as it does not interfere with the reaction, but preferably a buffer solution having a pH of 6 to 8 and more preferably a buffer solution having a pH of 6 to 7 and containing potassium chloride and sodium phosphate. Moreover, from the viewpoint of more easily promoting the reaction, it is preferable to contain iron sulfate.

The reaction temperature is not particularly limited either as long as the reaction is not hindered, but is usually 20 to 40° C. and preferably 25 to 37° C. Moreover, the reaction time is not particularly limited as long as it is a time for which the unsaturated hydrocarbon compound can be produced, but is usually 30 minutes to 7 days and preferably 12 hours to 2 days.

In addition, the second chain-shaped unsaturated carboxylic acid compound produced can be collected by appropriately using a known recovery and purification method (such as distillation and chromatography). Moreover, these methods may be carried out alone or may be carried out in multiple steps in appropriate combination.

Additionally, the first chain-shaped unsaturated carboxylic acid compound, which serves as a raw material, can be purchased as a commercially available product, as demonstrated in the subsequent examples. Furthermore, those skilled in the art could synthesize it while suitably referring to known synthesis methods (such as the method described in the production of L-lysine by fermentation (JP H 0530985 A)).

### (BesC)

“BesC” refers to an enzyme involved in β-ethynylserine (Bes) biosynthesis. This enzyme is a terminal alkene-producing enzyme that has an activity to catalyze a reaction that forms a carbon-carbon double bond at the terminus by oxidizing the propylamino group at the terminus to cleave the carbon-carbon bond within this group (NPL 2).

As for the origin of BesC, there are no particular restrictions as long as it has catalytic activity that promotes the production of the second chain-shaped unsaturated carboxylic acid compound, and various biologically derived ones can be used. For example, Pseudomonas fluorescens and Streptomyces cattleya can be mentioned as sources of BesC. Among these, BesC derived from Pseudomonas fluorescens (BesC composed of an amino acid sequence set forth in SEQ ID NO: 11) is preferable.

Additionally, the BesC according to the present invention has identity with the amino acid sequence set forth in SEQ ID NO: 11 by preferably 15% or more (for example, 16% or more, 17% or more, 18% or more, 19% or more), more preferably 20% or more (for example, 30% or more, 40% or more), further preferably 50% or more (for example, 60% or more, 70% or more), more preferably 80% or more (for example, 85% or more, 86% or more, 87% or more, 88% or more, 89% or more), and more preferably 90% or more (for example, 91% or more, 92% or more, 93% or more, 94% or more, 95% or more, 96% or more, 97% or more, 98% or more, 99% or more). Note that the term “identity” to the amino acid sequence set forth in SEQ ID NO: 11 means the proportion (%) of the number of amino acids that matches the amino acid sequence set forth in SEQ ID NO: 11 in the BesC according to the present invention, based on the total number of amino acids in the BesC according to the present invention.

Additionally, the BesC according to the present invention may be one in which mutations have been introduced naturally or unnaturally (artificially) into the amino acid sequence set forth in SEQ ID NO: 11. In other words, the BesC according to the present invention may also include proteins composed of amino acid sequences in which one or more amino acids in the amino acid sequence of BesC (such as the amino acid sequence set forth in SEQ ID NO: 11) have been substituted, deleted, added, and/or inserted. The term “more” here implies no particular restriction, but typically it is 2 to 80, preferably 2 to 70, more preferably 2 to 60, further preferably 2 to 50, more preferably 2 to 40, further preferably 2 to 30, more preferably 2 to 20, and further preferably 2 to 10 (for example, 2 to 9, 2 to 8, 2 to 7, 2 to 6, 2 to 5, 2 to 4, 2 or 3, or 2).

Note that whether BesC, or natural or non-natural mutants thereof, possess catalytic activity to produce a second chain-shaped unsaturated carboxylic acid compound can be determined by those skilled in the art through known methods (such as chromatography mass spectrometry) by directly measuring the quantity of the unsaturated carboxylic acid compound that is produced.

In addition, the BesC according to the present invention may be added with additional compounds directly or indirectly, as with the phenylalanine ammonia lyase according to the present invention described above. Furthermore, the BesC according to the present invention may be used by mixing with other components, as with phenylalanine ammonia lyase.

### <Method for Producing Chain-Shaped Unsaturated Hydrocarbon Compound>

As demonstrated in the following examples, the present inventors have found that it is also possible to produce a chain-shaped unsaturated hydrocarbon compound, which has a carbon-carbon double bond at both terminuses, from the above-mentioned third chain-shaped unsaturated carboxylic acid compound.

Specifically, the production method also takes the form indicated below, which includes the steps of producing a third chain-shaped unsaturated carboxylic acid compound, represented by the following formula (3), from the second chain-shaped unsaturated carboxylic acid compound represented by the following formula (2) using phenylalanine ammonia lyase as mentioned above, and producing a chain-shaped unsaturated hydrocarbon compound or a geometric isomer thereof, represented by the following formula (4) (hereinafter simply referred to as a “chain-shaped unsaturated hydrocarbon compound”), from the unsaturated carboxylic acid compound by using ferulic acid decarboxylase to decarboxylate carboxyl groups.

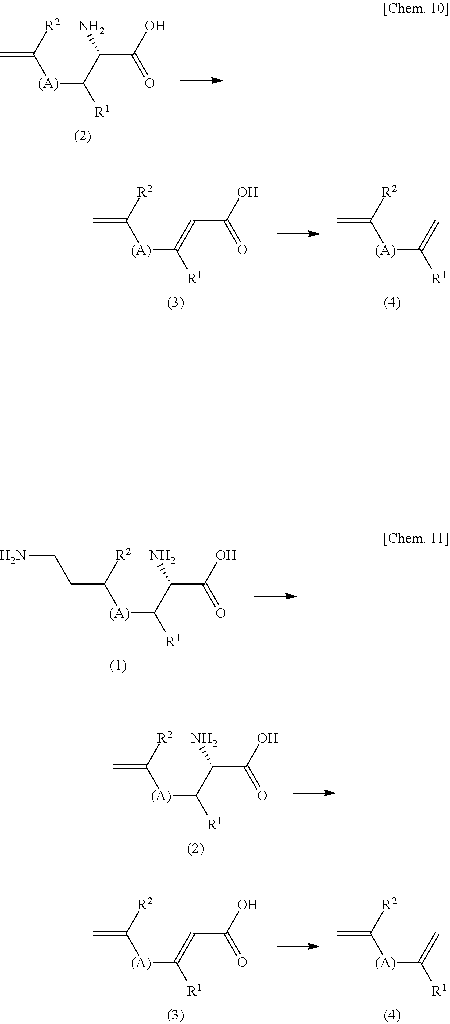

In the present invention, the “chain-shaped unsaturated hydrocarbon compound” produced in the aforementioned reaction means a chain-shaped unsaturated hydrocarbon compound or a geometric isomer thereof, represented by the formula (4), which has carbon-carbon double bonds at both terminuses.

In the compound, for (A) and R¹, including their preferable embodiments, they are as mentioned above, and the “chain-shaped unsaturated hydrocarbon compound” is preferably butadiene and isoprene.

It suffices that the condition of decarboxylating the third chain-shaped unsaturated carboxylic acid compound in the presence of ferulic acid decarboxylase according to the present invention is a condition in which the decarboxylation is promoted and an unsaturated hydrocarbon compound is produced. Those skilled in the art can appropriately adjust and set the composition of the reaction liquid, the pH of the reaction liquid, the reaction temperature, the reaction time, and the like.

For example, the reaction liquid added with the ferulic acid decarboxylase according to the present invention and its substrate, the unsaturated hydrocarbon dicarboxylic acid compound, is not particularly limited as long as it does not interfere with the reaction, but preferably a buffer solution having a pH of 6 to 8 and more preferably a buffer solution having a pH of 6 to 7 and containing potassium chloride and sodium phosphate. Moreover, from the viewpoint of more easily promoting the reaction, it is preferable to contain prenylated flavin mononucleotide (prFMN) or an isomer thereof (prFMN^{ketimine}, prFMN^{iminiu}, for these prFMN and isomers thereof, see NPL 1).

In addition, the reaction temperature is not particularly limited either as long as the reaction is not hindered, but is usually 20 to 40° C. and preferably 25 to 37° C. Moreover, the reaction time is not particularly limited as long as it is a time for which the unsaturated hydrocarbon compound can be produced, but is usually 30 minutes to 7 days and preferably 12 hours to 2 days.

In addition, the chain-shaped unsaturated hydrocarbon compound produced under such conditions is generally easily vaporized, and thus can be collected by a known volatile gas recovery and purification method. Examples of such collection method include gas stripping, fractional distillation, adsorption, desorption, pervaporation, desorption of unsaturated hydrocarbon compound adsorbed on the solid phase from the solid phase by heat or vacuum, extraction with a solvent, and chromatography (for example, gas chromatography). In addition, when the unsaturated hydrocarbon compound produced is a liquid, it can also be collected by appropriately using a known recovery and purification method (such as distillation and chromatography). Moreover, these methods may be carried out alone or may be carried out in multiple steps in appropriate combination.

### (Ferulic Acid Decarboxylase)

The “ferulic acid decarboxylase” is an enzyme registered as EC number: 4.1.1.102, and usually means an enzyme which catalyzes a reaction that decarboxylates ferulic acid to produce 4-vinylguaiacol (4VG).

In the present invention, there are no particular restrictions on the “ferulic acid decarboxylase” as long as it has the activity to catalyze the reaction that removes carboxyl groups from the third chain-shaped unsaturated carboxylic acid compound to produce a chain-shaped unsaturated hydrocarbon compound, and various biologically derived ones can be used. For example, proteins corresponding to “Ferulic acid decarboxylase” on UNIPROT as shown in Tables 1 to 6 of PTLs 3 can be mentioned. Also, as the ferulic acid decarboxylase according to the present invention, preferably Saccharomyces-derived ferulic acid decarboxylase and Aspergillus niger-derived ferulic acid decarboxylase (UNIPROT ID: A2QHE5 and the like) can be mentioned, more preferably Saccharomyces-derived ferulic acid decarboxylase can be mentioned. Note that “Saccharomyces” means bacteria belonging to the Saccharomyces genus, and examples thereof include Saccharomyces cerevisiae, Saccharomyces kudriavzevii, Saccharomyces eubayanus, Saccharomyces bayanus, Saccharomyces boulardii, Saccharomyces bulderi, Saccharomyces cariocanus, Saccharomyces cariocus, Saccharomyces chevalieri, Saccharomyces dairenensis, Saccharomyces ellipsoideus, Saccharomyces florentinus, Saccharomyces kluyveri, Saccharomyces martiniae, Saccharomyces monacensis, Saccharomyces norbensis, Saccharomyces paradoxus, Saccharomyces pastorianus, Saccharomyces spencerorum, Saccharomyces turicensis, Saccharomyces unisporus, Saccharomyces uvarum, and Saccharomyces zonatus.

As a ferulic acid decarboxylase derived from Saccharomyces, for example, FDC (UNIPROT ID: Q03034, composed of an amino acid sequence set forth in SEQ ID NO: 13) derived from Saccharomyces cerevisiae (ATCC 204508/S288c strain) (baker's yeast) can be mentioned, as well as proteins derived from Saccharomyces that correspond to “Ferulic acid decarboxylase” on UNIPROT, and more specifically, the ferulic acid decarboxylases shown in Table 1 below can be mentioned. Note that in nature, changes in the nucleotide sequence can cause changes in the amino acid sequence of the protein.

TABLE 1

| Uniprot_ID | Entry Name | Origin |
|---|---|---|
| Q03034 | FDC1_YEAST | Saccharomyces cerevisiae |
| | | (strain ATCC |
| | | 204508/8288c) |
| | | (Baker's yeast) |
| H0GEV9 | H0GEV9_SACCK | Saccharomyces cerevisiae |
| | | x Saccharomyces |
| | | kudiayzevii |
| | | (strain VIN7) |
| E9P8F2 | E9P8F2_YEASX | Saccharomyces cerevisiae |
| E9P8F1 | E9P8F1_YEASX | Saccharomyces cerevisiae |
| A6ZZC4 | A6ZZC4_YEAS7 | Saccharomyces cerevisiae |
| | | (strain YJM789) |
| N1P7L1 | N1P7L1_YEASC | Saccharomyces cerevisiae |
| | | strain CEN. PK113-7D |
| H0GPK3 | H0GTK3_SACCK | Saccharomyces cerevisiae |
| | | x Saccharomyces |
| | | Kudriavzeyii |
| | | (strain VIN7) |
| J8TRN5 | JSTRN5_SACK1 | Saccharomyces |
| | | kudriavzevii |
| | | (strain ATCC |
| | | MYA-4449/AS |
| | | 2.2408/CBS |
| | | 8840/NBRC 1802/ |
| | | NCYC 2889) |
| A0A0L8RE03 | A0A0L8RF03_SACEU | Saccharomyces eubayanus |

Additionally, the ferulic acid decarboxylase according to the present invention has identity with the amino acid sequence set forth in SEQ ID NO: 13 by preferably 15% or more (for example, 16% or more, 17% or more, 18% or more, 19% or more), more preferably 20% or more (for example, 30% or more, 40% or more), further preferably 50% or more (for example, 60% or more, 70% or more), more preferably 80% or more (for example, 85% or more, 86% or more, 87% or more, 88% or more, 89% or more), and more preferably 90% or more (for example, 91% or more, 92% or more, 93% or more, 94% or more, 95% or more, 96% or more, 97% or more, 98% or more, 99% or more). Note that the term “identity” to the amino acid sequence set forth in SEQ ID NO: 13 means the proportion (%) of the number of amino acids that matches the amino acid sequence set forth in SEQ ID NO: 13 in the ferulic acid decarboxylase according to the present invention, based on the total number of amino acids in the ferulic acid decarboxylase according to the present invention.

Additionally, the ferulic acid decarboxylase according to the present invention may be one in which mutations have been introduced into the amino acid sequence set forth in SEQ ID NO: 13. In other words, the ferulic acid decarboxylase according to the present invention may also include proteins composed of amino acid sequences in which one or more amino acids in the amino acid sequence of ferulic acid decarboxylase (such as the amino acid sequence set forth in SEQ ID NO: 13) have been substituted, deleted, added, and/or inserted. The term “more” here implies no particular restriction, but typically it is 2 to 150, preferably 2 to 100, more preferably 2 to 80, further preferably 2 to 50, more preferably 2 to 30, further preferably 2 to 20, and more preferably 2 to 10 (for example, 2 to 9, 2 to 8, 2 to 7, 2 to 6, 2 to 5, 2 to 4, 2 or 3, or 2). Additionally, such mutants of ferulic acid decarboxylase may occur naturally or unnaturally (artificially). In other words, this includes ferulic acid decarboxylase variants where amino acid substitutions and the like have been artificially introduced.

Note that whether ferulic acid decarboxylase, or natural or non-natural mutants thereof, possess catalytic activity to produce chain-shaped unsaturated hydrocarbon compounds can be determined by those skilled in the art, for example, as demonstrated in the following examples, by directly measuring the amount of unsaturated hydrocarbon compounds using gas chromatography-mass spectrometry (GC-MS).

In addition, the ferulic acid decarboxylase according to the present invention may be added with additional compounds directly or indirectly, as with the phenylalanine ammonia lyase according to the present invention described above. Furthermore, the ferulic acid decarboxylase according to the present invention may be used by mixing with other components, as with phenylalanine ammonia lyase.

### <DNA Encoding Enzyme According to Present Invention and Vector Having the DNA>

Next, an explanation will be given regarding DNA and such that encodes the enzymes according to the present invention (phenylalanine ammonia lyase according to the present invention, BesC according to the present invention, and ferulic acid decarboxylase according to the present invention). By introducing such DNA, it is possible to transform the traits of the host cell, and produce various enzymes according to the present invention in that cell, thus enabling the production of a third chain-shaped unsaturated carboxylic acid compound, or a chain-shaped unsaturated hydrocarbon compound.

The DNA according to the present invention may be, as long as it encodes the above-mentioned enzyme according to the present invention, a natural DNA, a DNA obtained by artificially introducing a mutation into a natural DNA, or a DNA composed of an artificially designed nucleotide sequence. Moreover, the form thereof is not particularly limited, and includes a cDNA as well as a genomic DNA and a chemically synthesized DNA. These DNAs can be prepared by those skilled in the art using conventional means. The genomic DNA can be prepared, for example, as follows. Specifically, a genomic DNA is extracted from various bacteria to create a genomic library (as a vector, plasmid, phage, cosmid, BAC, PAC, or the like can be used). This is developed, and colony hybridization or plaque hybridization is performed using a probe prepared based on the nucleotide sequence of the gene encoding the enzyme according to the present invention (for example, the nucleotide sequence set forth in SEQ ID NO: 1). In addition, it is also possible to prepare a genomic DNA by preparing a primer specific to the gene encoding the enzyme according to the present invention and performing PCR using this primer. In addition, a cDNA can be prepared, for example, as follows. Specifically, an mRNA extracted from various bacteria is used as a basis to synthesize a cDNA, which is inserted into a vector such as AZAP to prepare a cDNA library. This is developed, and colony hybridization or plaque hybridization, or PCR is performed in the same manner as described above.

Then, those skilled in the art can introduce, if necessary, a mutation encoding the above-mentioned amino acid substitution into the DNA thus prepared by using a known site-specific mutagenesis. Examples of site-specific mutagenesis include the Kunkel method (Kunkel, T. A., Proc Natl Acad Sci USA, 1985, Volume 82, issue 2, pages 488 to 492) and the SOE (splicing-by-overlap-extension)-PCRmethod (Ho, S. N., Hunt, H. D., Horton, R. M., Pullen, J. K., and Pease, L. R., Gene, 1989, Volume 77, pages 51 to 59).

In addition, those skilled in the art can artificially design a nucleotide sequence encoding a protein in which the above-mentioned amino acid substitution has been introduced, and use an automatic nucleic acid synthesizer based on the sequence information to chemically synthesize the DNA according to the present invention.

Moreover, from the viewpoint of further improving the expression efficiency of the encoded decarboxylase according to the present invention in the host cell, the DNA according to the present invention may can the form of a DNA encoding the enzyme according to the present invention in which the codon is optimized in accordance with the type of the host cell.

In addition, in order to make it possible to replicate the above-mentioned DNA in the host cell, the present invention can take the form of a vector inserted with the DNA.

In the present invention, the “vector” can be constructed based on, for example, a plasmid, which exists as a self-replicating vector, that is, an extrachromosomal independent entity whose replication is independent of chromosomal replication. In addition, a vector may be one that, when introduced into a host cell, is integrated into the genome of the host cell and replicated together with the chromosome into which it has been integrated.

Examples of such vector include plasmids and phage DNAs. In addition, examples of the plasmids include plasmids derived from E. coli (such as pET22, pBR322, pBR325, pUC118, pUC119, pUC18, and pUC19), plasmids derived from yeast (such as YEp13, YEp24, and YCp50), and plasmids derived from Bacillus subtilis (such as pUB110 and pTP5). Examples of the phage DNAs include λ phages (such as Charon4A, Charon21A, EMBL3, EMBL4, λgt10, λgt11, and λZAP). Furthermore, as a vector according to the present invention, an insect virus vector such as a baculovirus can be used when the host cell is derived from an insect, T-DNA or the like can be used when the host cell is derived from a plant, and an animal virus vector such as a retrovirus or an adenovirus vector can also be used when the host cell is derived from an animal. In addition, as a vector construction procedure and method according to the present invention, ones conventionally used in the field of genetic engineering can be used. For example, in order to insert the DNA according to the present invention into a vector, a method is employed in which a purified DNA is first cleaved with an appropriate restriction enzyme, inserted into a restriction enzyme site or a multicloning site of an appropriate vector, and ligated to the vector.

In addition, the vector according to the present invention may be in the form of an expression vector containing the enzyme according to the present invention encoded by the DNA in a state capable of expression in a host cell. For the purpose of introduction into a host cell to express the enzyme according to the present invention, the “expression vector” according to the present invention preferably contains, in addition to the DNA, a DNA sequence for controlling the expression, a genetic marker for selecting a transformed host cell, and the like. Examples of the DNA sequence for controlling expression include promoters, enhancers, splicing signals, poly A addition signals, ribosome binding sequences (SD sequences), and terminators. The promoters are not particularly limited as long as they exhibit transcriptional activity in the host cell, and can be obtained as a DNA sequence which controls the expression of a gene encoding a protein that is of a type same as or different from the host cell. In addition to the DNA sequence which controls expression, a DNA sequence which induces expression may be contained. When the host cell is a bacterium, examples of such DNA sequence which induces expression include the lactose operon, which can induce expression of a gene located downstream by the addition of isopropyl-β-D-thiogalactopyranoside (IPTG). The gene marker in the present invention may be appropriately selected according to the method of selecting a transformed host cell. For example, it is possible to use a gene encoding drug resistance and a gene complementary to auxotrophy.

The vector may be inserted with a single type of DNA encoding the enzyme according to the present invention, and it is also possible to insert multiple types of DNA into one vector. In the case of inserting multiple types of DNA into a vector, when multiple types of DNA are inserted into one vector, it is preferable for these DNAs to form an operon. Here, “operon” refers to a nucleic acid sequence unit composed of one or more genes transcribed under the control of the same promoter.

In the present invention, examples of such vectors include the following:
a vector into which the DNA encoding the phenylalanine ammonia lyase according to the present invention has been inserted,
a combination of a vector into which the DNA encoding the BesC according to the present invention has been inserted, and a vector into which the DNA encoding the phenylalanine ammonia lyase according to the present invention has been inserted,
a combination of a vector into which the DNA encoding the phenylalanine ammonia lyase according to the present invention has been inserted, and a vector into which the DNA encoding the ferulic acid decarboxylase according to the present invention has been inserted, or
a combination of a vector into which the DNA encoding the BesC according to the present invention has been inserted, a vector into which the DNA encoding the phenylalanine ammonia lyase according to the present invention has been inserted, and a vector into which the DNA encoding the ferulic acid decarboxylase according to the present invention has been inserted.

In addition, it is also possible to mention the following:
a vector into which the DNA encoding the BesC according to the present invention and the DNA encoding the phenylalanine ammonia lyase according to the present invention have been inserted,
a vector into which the DNA encoding the phenylalanine ammonia lyase according to the present invention and the ferulic acid decarboxylase according to the present invention have been inserted, or
a vector into which the DNA encoding the BesC according to the present invention, the DNA encoding the phenylalanine ammonia lyase according to the present invention and the ferulic acid decarboxylase according to the present invention have been inserted.

In addition, the DNA or vector according to the present invention may be used by being mixed with additional components. The additional components are not particularly limited, and examples thereof include sterilized water, physiological saline, vegetable oil, surfactants, lipids, solubilizers, buffer agents, DNase inhibitors, and preservatives.

### <Agent for Promoting Production of Third Chain-Shaped Unsaturated Carboxylic Acid Compound or Chain-Shaped Unsaturated Hydrocarbon Compound>

As described above, it becomes possible to facilitate the production of the third chain-shaped unsaturated carboxylic acid compound, or chain-shaped unsaturated hydrocarbon compounds, by using the enzyme according to the present invention, the DNA encoding this enzyme, or the vector into which this DNA has been inserted.

Therefore, the present invention provides an agent for promoting the production of a third chain-shaped unsaturated carboxylic acid compound or a chain-shaped unsaturated hydrocarbon compound, comprising an enzyme according to the present invention, a DNA encoding the enzyme, or a vector into which the DNA has been inserted.

More specifically, the following embodiments can be mentioned:
an agent for promoting the production of a third chain-shaped unsaturated carboxylic acid compound from a second chain-shaped unsaturated carboxylic acid compound, comprising the phenylalanine ammonia lyase according to the present invention, a DNA encoding the enzyme, or a vector into which the DNA has been inserted,
an agent for promoting the production of a third chain-shaped unsaturated carboxylic acid compound from a first chain-shaped unsaturated carboxylic acid compound, comprising the BesC according to the present invention, a DNA encoding the enzyme, or a vector into which the DNA has been inserted, and the phenylalanine ammonia lyase according to the present invention, a DNA encoding the enzyme, or a vector into which the DNA has been inserted,
an agent for promoting the production of a third chain-shaped unsaturated carboxylic acid compound from a first chain-shaped unsaturated carboxylic acid compound, comprising a vector into which a DNA encoding the BesC according to the present invention and a DNA encoding the phenylalanine ammonia lyase according to the present invention have been inserted,
an agent for promoting the production of a chain-shaped unsaturated hydrocarbon compound from a second chain-shaped unsaturated carboxylic acid compound, comprising:
   the phenylalanine ammonia lyase according to the present invention, a DNA encoding the enzyme, or a vector into which the DNA has been inserted; and
   the ferulic acid decarboxylase according to the present invention, a DNA encoding the enzyme, or a vector into which the DNA has been inserted,
an agent for promoting the production of a chain-shaped unsaturated hydrocarbon compound from a second chain-shaped unsaturated carboxylic acid compound, comprising a vector into which a DNA encoding the phenylalanine ammonia lyase according to the present invention and a DNA encoding the ferulic acid decarboxylase according to the present invention have been inserted,
an agent for promoting the production of a chain-shaped unsaturated hydrocarbon compound from a first chain-shaped unsaturated carboxylic acid compound, comprising:
   the BesC according to the present invention, a DNA encoding the enzyme or a vector into which the DNA has been inserted; the phenylalanine ammonia lyase according to the present invention, a DNA encoding the enzyme or a vector into which the DNA has been inserted; and
   the ferulic acid decarboxylase according to the present invention, a DNA encoding the enzyme or a vector into which the DNA has been inserted,
an agent for promoting the production of a chain-shaped unsaturated hydrocarbon compound from a first chain-shaped unsaturated carboxylic acid compound, comprising a vector into which a DNA encoding the BesC according to the present invention, a DNA encoding the phenylalanine ammonia lyase according to the present invention and a DNA encoding the ferulic acid decarboxylase according to the present invention have been inserted.

Such an agent may be one containing the enzyme according to the present invention or the like, or may be used by being mixed with additional components. Such additional components are not particularly limited, and examples thereof include sterilized water, physiological saline, vegetable oil, surfactants, lipids, solubilizers, buffer agents, protease inhibitors, DNase inhibitors, and preservatives.

The present invention can also provide a kit including such an agent. In the kit of the present invention, the agent may be included in the form of a host cell described later into which the DNA or the like according to the present invention has been introduced and transformed. Moreover, in addition to such an agent, the kit of the present invention may include various substrates (the first chain-shaped unsaturated carboxylic acid compound and the second chain-shaped unsaturated carboxylic acid compound), a host cell for introducing the DNA or the like according to the present invention, a medium for culturing the host cell, an instruction manual for use thereof, and the like. Moreover, such an instruction manual is a manual for using the agent of the present invention and the like in the method for producing the above-mentioned third chain-shaped unsaturated carboxylic acid compound or chain-shaped unsaturated hydrocarbon compound. The manual can include, for example, information on the experimental method and experimental conditions for the production method of the present invention, the agent of the present invention, and the like (for example, information such as a vector map showing the nucleotide sequence of a vector, sequence information on the enzyme according to the present invention, information on the origin and properties of the host cell as well as the culture conditions of the host cell, and the like).

<Host Cell Introduced with DNA Encoding Enzyme According to Present Invention, and the Like>

Next, description is provided for a host cell introduced with the DNA or vector according to the present invention. Use of a host cell transformed by the introduction of the above-mentioned DNA or vector makes it possible to produce the enzyme according to the present invention, and makes it possible to further produce the third chain-shaped unsaturated carboxylic acid compound or chain-shaped unsaturated hydrocarbon compound.

The host cell introduced with the DNA or vector according to the present invention is not particularly limited, and examples thereof include microorganisms (such as E. coli, budding yeast, fission yeast, Bacillus subtilis, actinomycetes, and filamentous fungi), plant cells, insect cells, and animal cells. However, it is preferable to use a microorganism, and it is more preferable to use E. coli as a host cell from the viewpoint that it is possible to contribute to the production of the third chain-shaped unsaturated carboxylic acid compound or chain-shaped unsaturated hydrocarbon compound with a relatively inexpensive medium, with high proliferation properties in a short time, and further with high productivity.

In addition, the host cell introduced with the DNA or vector according to the present invention is preferably a cell which retains a flavin prenyltransferase from the viewpoint of inducing prenylation of flavin mononucleotide (FMN) and producing prFMN or an isomer thereof which contributes to improving the productivity of the chain-shaped unsaturated hydrocarbon compound.

In addition, the host cell introduced with the DNA or vector according to the present invention is preferably E. coli C41 (DE3) cells because they are easy to handle and can express toxic proteins.

The introduction of the DNA or vector according to the present invention can be carried out according to a method conventionally used in this field. Examples of the method for introduction into microorganisms such as E. coli include the heat shock method, the electroporation method, the spheroplast method, and the lithium acetate method, examples of the method for introduction into plant cells include a method using Agrobacterium and the particle gun method, examples of the method for introduction into insect cells include a method using baculovirus and the electroporation method, and examples of the method for introduction into animal cells include the calcium phosphate method, the lipofection method, and the electroporation method.

The DNA or the like thus introduced into the host cell may be, in the host cell, retained by being randomly inserted into its genomic DNA, may be retained by homologous recombination, and can be, in the case of a vector, replicated and retained as an independent entity outside the genomic DNA.

### <Method for Producing Third Chain-Shaped Unsaturated Carboxylic Acid Compound or Chain-Shaped Unsaturated Hydrocarbon Compound Using Host Cell>

As described above, by cultivating a host cell that has been transformed to express an enzyme according to the present invention, it is possible to produce a third chain-shaped unsaturated carboxylic acid compound or chain-shaped unsaturated hydrocarbon compound. Therefore, the present invention also provides a method for producing a third chain-shaped unsaturated carboxylic acid compound or chain-shaped unsaturated hydrocarbon compound, including the steps of cultivating a host cell into which a DNA or vector encoding an enzyme according to the present invention has been introduced, and collecting the third chain-shaped unsaturated carboxylic acid compound or chain-shaped unsaturated hydrocarbon compound that has been produced in the host cell and/or a culture thereof.

“The host cell that has been transformed to express an enzyme according to the present invention” is as described above. Furthermore, in such host cells, the combination of the enzyme according to the present invention to be expressed or retained, the DNA encoding the enzyme, and the vector into which the DNA has been inserted, can be subjected to appropriate design changes by those skilled in the art in consideration of the combination of starting materials (substrates) and final products based on the examples shown in the aforementioned <DNA Encoding Enzyme According to Present Invention and Vector Having the DNA> and <Agent for Promoting Production of Third Chain-Shaped Unsaturated Carboxylic Acid Compound or Chain-Shaped Unsaturated Hydrocarbon Compound>.

The culture conditions for the cell are as described later, and the medium is preferably added with various substrates (the first chain-shaped unsaturated carboxylic acid compound and the second chain-shaped unsaturated carboxylic acid compound). The culture temperature can be appropriately changed according to the type of the host cell to be used, but is usually 20 to 40° C. and preferably 25 to 37° C.

In the present invention, the “culture” is a medium containing proliferated host cells, secreted products of the host cells, metabolites of the host cells, and the like obtained by culturing the host cells in a medium, and includes dilutions and concentrates thereof. The collection of a third chain-shaped unsaturated carboxylic acid compound or chain-shaped unsaturated hydrocarbon compound from such host cell and/or culture is not particularly limited either, and can be performed using the above-described known recovery and purification methods. In addition, the time period of collection is appropriately adjusted according to the type of the host cell to be used, and may be any time which can produce a third chain-shaped unsaturated carboxylic acid compound or chain-shaped unsaturated hydrocarbon compound, but is usually 30 minutes to 7 days and preferably 12 hours to 2 days.

### <Method for Producing Phenylalanine Ammonia Lyase Variant According to Present Invention>

As presented in Examples to be described later, culturing of a host cell introduced with a DNA or the like encoding the phenylalanine ammonia lyase variant according to the present invention makes it possible to produce the variant in the host cell.

Therefore, the present invention can provide a method for producing a phenylalanine ammonia lyase variant, including culturing a host cell introduced with a DNA encoding the phenylalanine ammonia lyase variant according to the present invention or a vector containing the DNA, and collecting a protein expressed in the host cell.

In the present invention, the conditions for “culturing a host cell” may be any conditions as long as the host cell can produce the phenylalanine ammonia lyase variant according to the present invention, and according to the type of the host cell, the medium used, and the like, those skilled in the art can appropriately adjust and set the temperature, the presence or absence of addition of air, the concentration of oxygen, the concentration of carbon dioxide, the pH of the medium, the culture temperature, the culture time, the humidity, and the like.

Such a medium only needs to have a content which can be used as a nutrient source by the host cell, and examples of the content include carbon sources, nitrogen sources, sulfur sources, inorganic salts, metals, peptones, yeast extracts, meat extracts, casein hydrolysates, and serum. In addition, such a medium may be added with, for example, IPTG for inducing the expression of a DNA encoding the phenylalanine ammonia lyase variant according to the present invention, an antibiotic corresponding to the drug resistance gene which can be encoded by the vector according to the present invention (for example, ampicillin), or a nutrient corresponding to a gene complementing the auxotrophy which can be encoded by the vector according to the present invention (for example, arginine or histidine).

Additionally, examples of the method for “collecting a protein expressed in the host cell” from the host cell thus cultured include a method in which the host cell is recovered from the medium by filtration, centrifugation, or the like, the recovered host cell is treated by cell lysis, grinding treatment, or pressure crushing, and further, the protein expressed in the host cell is purified and concentrated by ultrafiltration treatment, salting out, solvent precipitation such as ammonium sulfate precipitation, chromatography (such as gel chromatography, ion exchange chromatography, or affinity chromatography), or the like. Moreover, when the phenylalanine ammonia lyase variant according to the present invention is added with the above-mentioned purified tag protein, it can be purified and collected using a substrate to which the tag protein is adsorbed. Moreover, these purification and concentration methods may be carried out alone or may be carried out in multiple steps in appropriate combination.

In addition, the phenylalanine ammonia lyase variant according to the present invention is not limited to the above-described biological synthesis, and can also be produced using the DNA or the like according to the present invention and a cell-free protein synthesis system. Such a cell-free protein synthesis system is not particularly limited, and examples thereof include synthesis systems derived from wheat germ, E. coli, rabbit reticulocytes, and insect cells. Moreover, those skilled in the art can chemically synthesize the phenylalanine ammonia lyase variant according to the present invention using a commercially available peptide synthesizer or the like.

In addition, the present invention provides a method for producing a phenylalanine ammonia lyase variant with enhanced catalytic activity to produce a third chain-shaped unsaturated carboxylic acid compound from a second chain-shaped unsaturated carboxylic acid compound, the production method including introducing at least one of the following amino acid substitutions (1) to (5) in phenylalanine ammonia lyase:
(1) substituting position 108 in the amino acid sequence set forth in SEQ ID NO: 2, or an amino acid corresponding to the position, with methionine, phenylalanine, or valine,
(2) substituting position 107 in the amino acid sequence set forth in SEQ ID NO: 2, or an amino acid corresponding to the position, with tryptophan,
(3) substituting position 219 in the amino acid sequence set forth in SEQ ID NO: 2, or an amino acid corresponding to the position, with isoleucine,
(4) substituting position 223 in the amino acid sequence set forth in SEQ ID NO: 2, or an amino acid corresponding to the position, with isoleucine,
(5) substituting position 104 in the amino acid sequence set forth in SEQ ID NO: 2, or an amino acid corresponding to the position, with alanine.

The term “phenylalanine ammonia lyase variant with enhanced catalytic activity to produce a third chain-shaped unsaturated carboxylic acid compound from a second chain-shaped unsaturated carboxylic acid compound” means phenylalanine ammonia lyase with higher catalytic activity to produce a third chain-shaped unsaturated carboxylic acid compound compared to before the introduction of the amino acid substitution, and the comparison target typically includes various phenylalanine ammonia lyases derived from different organisms and natural mutants thereof.

The introduction of “amino acid substitution” in phenylalanine ammonia lyase can be accomplished by modifying the DNA that encodes it. Such “DNA modification” can be appropriately carried out, as mentioned above, by those skilled in the art using well-known methods such as site-specific mutagenesis and chemical synthesis of DNA based on modified sequence information. Additionally, the introduction of “amino acid substitution” can also be carried out using the chemical synthesis of peptides as described above.

Whether the introduction of such amino acid substitution enhances the catalytic activity for the production of olefin compounds can be assessed, as mentioned earlier, by chromatography mass spectrometry, and other methods. Moreover, phenylalanine ammonia lyase variants produced in this manner have a catalytic activity to produce a third chain-shaped unsaturated carboxylic acid compound, which is preferably 1.2 times or more (1.3 times or more, 1.4 times or more, 1.5 times or more, 1.6 times or more, 1.7 times or more, 1.8 times or more, 1.9 times or more), more preferably 2 times or more, further preferably 3 times or more, more preferably 4 times or more, and further preferably 5 times or more or more, as compared to the phenylalanine ammonia lyase composed of the amino acid sequence set forth in SEQ ID NO: 2.

### EXAMPLES

Hereinafter, the present invention is described more specifically based on Examples, but the present invention is not limited to the following Examples.

The present inventors envisioned the following reaction scheme to produce butadiene with L-lysine as a starting material in a three-step reaction using three enzymes (BesC, PAL, FDC).

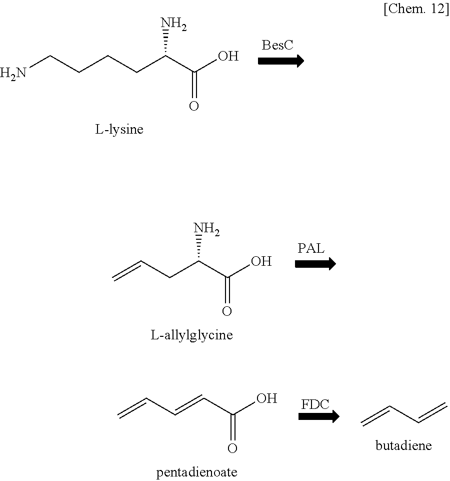

Therefore, a demonstration experiment was carried out for this reaction scheme as follows.

### Example 1

### <Preparation of Plasmid Vector>

First, in order to efficiently express BesC derived from Pseudomonas fluorescens, PAL derived from Arabidopsis thaliana, and FDC derived from Saccharomyces cerevisiae in E. coli, the frequency of codon usage in E. coli was considered to perform modification, thereby designing a nucleotide sequence. Subsequently, a DNA composed of such a modified nucleotide sequence was chemically synthesized according to a conventional method. Then, the DNA thus prepared and the pET22b(+) vector (manufactured by Novagen) were ligated by the Gibson Assembly method (using a kit of New England Biolabs, NEBuilder HiFi DNA Assembly Master Mix (registered trademark)), to thereby prepare plasmid vectors (BesC vector, PAL vector, FDC vector 1) capable of expressing the various wild type genes in E. coli. Similarly, the pColADuet vector (manufactured by Novagen) and a DNA obtained by amplifying a gene (SEQ ID NO: 15) encoding flavin prenyltransferase (hereinafter also referred to as “UbiX”) from E. coli (K-12) strain by the PCR method were ligated by the Gibson Assembly method, to thereby prepare a plasmid vector (UbiX vector) capable of expressing the wild type UbiX in E. coli.

Note that for the amino acid sequences of the enzymes used in Example 1 and the subsequent Example 2, and the DNA sequences encoding them (wild type sequences, sequences modified considering the codon usage frequency in E. coli), refer to the sequences set forth in the SEQ ID NOs shown in Table 2 below.

TABLE 2

| | | | DNA Sequence |
|---|---|---|---|
| | Amino Acid | Wild Type | with |
| | Sequence | DNA Sequence | Optimized Codon |
| AvPAL | SEQ ID NO: 2 | SEQ ID NO: 1 | SEQ ID NO: 3 |
| PaPaL | SEQ ID NO: 5 | SEQ ID NO: 4 | SEQ ID NO: 6 |
| AtPAL | SEO ID NO: 8 | SEQ ID NO: 7 | SEQ ID NO: 9 |
| PfBesC | SEQ ID NO: 11 | — | SEQ ID NO: 10 |
| ScFDC | SEQ ID NO: 13 | SEQ ID NO: 12 | SEQ ID NO: 14 |
| UbiX | SEQ ID NO: 16 | SEQ ID NO: 15 | — |

### <Preparation of Enzyme Expression Medium and Measurement of Enzyme Activity>

The vectors prepared as described above (5 μg of BesC vector or 5 μg of PAL vector) were introduced into E. coli C41 (DE3) strain (manufactured by Lucigen Corporation, 100 μL) by the heat shock method to prepare transformants expressing wild type BesC or PAL. Then, each of these transformants was cultured for 6 hours in an LB medium supplemented with ampicillin. It should be noted that the growth of these transformants reaches a peak after such 6-hour culture (pre-culture). For this reason, the amount of bacterial cells at the start of the enzyme reaction to be described later is uniform among these transformants.

In addition, addition was carried out to achieve 12 g/L tryptone, 24 g/L yeast extract, 10 g/L glycerol, 9.4 g/L dipotassium hydrogen phosphate, 2.2 g/L potassium dihydrogen phosphate, 20 g/L lactose, and 100 mg/L ampicillin, to thereby prepare an enzyme expression medium.

In a 500 mL baffled triangular flask, 1 mL of the aforementioned E. coli culture fluid that had been cultivated for 6 hours, and 100 mL of the aforementioned enzyme expression medium were added, followed by further cultivation at 37° C. and a shaking rate of 180 rpm for an additional 18 hours. After cultivation, the culture medium was centrifuged at 5,000 rpm for 10 minutes, and after the supernatant was removed, 10 mL of protein extraction solution B-PER™ Bacterial Cell Lysis Reagent (Thermo Fisher Scientific) was added to suspend the pellet. After shaking it at a permeation speed of 60 rpm in an ice bath, the various cell lysates were prepared by centrifuging it at 15,000 rpm for 15 minutes.

Similarly, the prepared vectors (5 μg of FDC vector 1 and 5 μg of UbiX vector) were introduced into E. coli C41 (DE3) strain (manufactured by Lucigen Corporation, 100 μL) by the heat shock method to prepare transformants co-expressing wild type FDC and UbiX. They were cultured for 6 hours in LB medium supplemented with ampicillin and kanamycin. The above enzyme reaction medium supplemented with 50 mg/L kanamycin was prepared, and a cell lysate was prepared in the same manner.

Then, to a 10 mL vial for a headspace type gas chromatography mass spectrometer (HS/GSMS), 100 μL each of the various cell lysates, 100 μL of 1 M phosphate buffer solution (pH 6.8), L-lysine to a final concentration of 10 mM, and ultrapure water to 1 mL were added. Immediately after that, the cap of the vial was closed, followed by enzyme reaction at 37° C. and a shaking rate of 180 rpm. The peak areas representing the amount of 1,3-butadiene produced in the headspace of the vial 18 hours after the start of the reaction were measured by GC-MS (manufactured by Shimadzu Corporation under the trade name: GCMS-QP Ultra).

As a result, although not shown in the figure, butadiene was not detected without the addition of L-lysine, but the production of 1,3-butadiene was confirmed with the addition of L-lysine.

### Example 2

As shown in Example 1, the combination of various wild type enzymes was observed to produce butadiene from L-lysine. Therefore, in order to further improve this catalytic activity, PALs derived from other hosts were examined for the PALs that perform the second step in the reaction.

### <Preparation of Plasmid Vector>

In order to efficiently express PAL derived from Anabaena variabilis or Plagiochasma appendiculatum in E. coli, the frequency of codon usage in E. coli was considered to perform modification, thereby designing a nucleotide sequence. Subsequently, a DNA composed of such a modified nucleotide sequence was chemically synthesized according to a conventional method, to thereby prepare plasmid vectors (various PAL vectors) capable of expressing the various wild type genes in E. coli, as in the same manner as above.

Subsequently, the pZA33luc vector (manufactured by Expressys) and a DNA obtained by amplifying FDC vector 1 and pTrcHis B vector (Thermo Fisher Scientific) by the PCRmethod were ligated by the Gibson Assembly method, to thereby prepare an FDC plasmid vector (FDC vector 2) for enzyme activity measurement in E. coli.

### <Preparation of Enzyme Activity Measurement Medium and Measurement of Enzyme Activity>

The prepared vectors (5 μg of PAL vector, 5 μg of UbiX vector, and 5 μg of FDC vector 2) were introduced into E. coli C41 (DE3) strain (manufactured by Lucigen Corporation, 100 μL) by the heat shock method to prepare a transformant co-expressing various PALs and wild type FDC and UbiX.

Then, each of these transformants was cultured for 6 hours in an LB medium supplemented with ampicillin, kanamycin, and chloramphenicol. It should be noted that the growth of these transformants reaches a peak after such 6-hour culture (pre-culture). For this reason, the amount of bacterial cells at the start of the enzyme reaction to be described later is uniform among these transformants.

Further, in addition to 12 g/L tryptone, 24 g/L yeast extract, 10 g/L glycerol, 9.4 g/L dipotassium hydrogen phosphate, 2.2 g/L potassium dihydrogen phosphate, 20 g/L lactose, 100 mg/L ampicillin, 50 mg/L kanamycin, and 30 mg/L chloramphenicol, 1 mM phenylalanine or 1 mM allylglycine was added as a substrate to prepare an enzyme activity measurement medium.

Then, to a 10 mL vial for HS/GSMS, 50 μL of culture solution of the aforementioned transformants and 1 mL of enzyme activity measurement medium were added, and immediately after that, the cap of the vial was closed, followed by enzyme reaction at 37° C. and a shaking rate of 180 rpm. The peak areas representing the amount of styrene or 1,3-butadiene produced in the headspace of the vial 18 hours after the start of the reaction were measured by GC-MS.

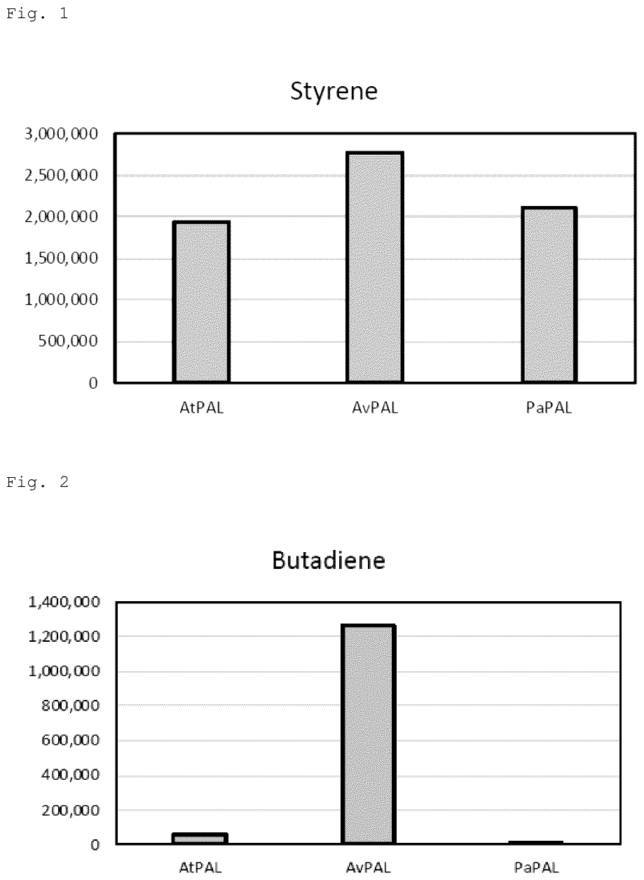

As a result, as shown in FIG. 1, when phenylalanine was added as a substrate, styrene production was observed for all PALs, as indicated below. Moreover, no significant difference was seen in its amount produced between the three types of PALs.

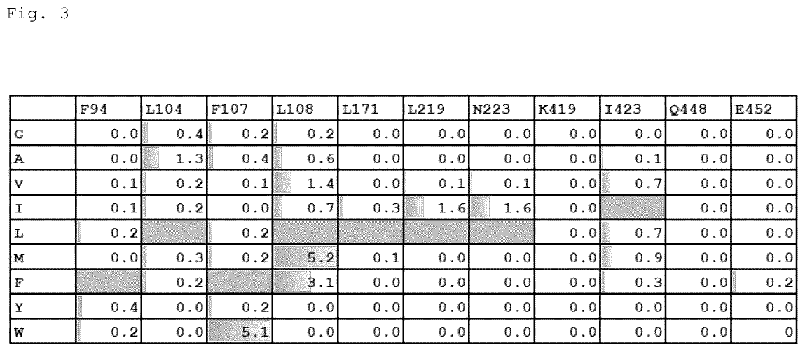

On the other hand, as shown in FIG. 2, when allylglycine was added as a substrate, butadiene production was observed from all PALs. However, a significant difference was observed in the amount of butadiene produced, with the highest butadiene production confirmed when using AvPAL.

### Example 3

As demonstrated in Example 2, high catalytic activity for the production of butadiene was observed in the wild type AvPAL. Therefore, in order to further enhance this high catalytic activity, mutations were introduced that replaced the enzyme active sites with different amino acids. The catalytic activity of the resulting mutants was then evaluated.

Specifically, primers encoding amino acid sequences with those mutations introduced were designed and synthesized. Then, using the vector encoding the wild-type AvPAL prepared in Example 2 as a template, plasmid vectors (PAL variant vectors) were prepared capable of expressing PALs with introduced mutations in E. coli, using the aforementioned primers in accordance with the Gibson Assembly protocol. Then, in the above <Preparation of Enzyme Activity Measurement Medium and Measurement of Enzyme Activity>, instead of the wild-type PAL vector, the PAL variant vectors were introduced, transformants were prepared, and the enzyme activity was measured. With the butadiene production quantity when using the wild-type PAL set as 1.0, the butadiene production quantity when using the PAL variant is shown in FIG. 3.

As shown in FIG. 3, the introduction of a variety of amino acid substitutions resulted in an observed increase in butadiene production quantity compared to the wild type before the introduction, in the following amino acid substitutions:
(1) the leucine at position 108 was substituted with methionine, phenylalanine, or valine,
(2) the phenylalanine at position 107 was substituted with tryptophan,
(3) the leucine at position 219 was substituted with isoleucine,
(4) the asparagine at position 223 was substituted with isoleucine,
(5) the leucine at position 104 was substituted with alanine.

### INDUSTRIAL APPLICABILITY

As described above, the present invention makes it possible to provide a method for producing an unsaturated compound having at least two carbon-carbon double bonds using an enzyme. For example, the present invention also makes it possible to produce butadiene using relatively inexpensive L-lysine as a starting material. Additionally, according to the present invention, unsaturated compounds can be produced by biosynthesis rather than by chemical synthesis, thus reducing environmental impact. Therefore, the present invention is extremely useful in the production of raw materials, including butadiene, for various synthetic polymers such as synthetic rubber.

1. A method for producing a chain-shaped unsaturated carboxylic acid compound or a geometric isomer thereof, represented by the following formula (3), comprising the steps of: removing a first amino group from a chain-shaped unsaturated carboxylic acid compound or a geometric isomer thereof, having a first amino group and a first carbon-carbon double bond at a terminus, represented by the following formula (2), in the presence of phenylalanine ammonia lyase, and forming a second carbon-carbon double bond 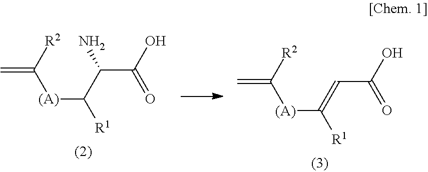
[in the formulas, (A) represents a linear hydrocarbon group having 0 to 5 carbon atoms, which may be substituted, and when the number of carbon atoms is 2 to 5, it may form a double bond between adjacent carbon atoms. R¹ and R² each independently represent a hydrogen atom, a linear or branched alkyl group having 1 to 5 carbon atoms, a linear or branched alkoxy group having 1 to 5 carbon atoms, or a hydroxy group.].

2. The method according to claim 1, further comprising the steps of: removing a second amino group and a methylene group from a chain-shaped carboxylic acid compound or a geometric isomer thereof, having a first amino group and a second amino group at a terminus, represented by the following formula (1), in the presence of the terminal alkene generating enzyme BesC, and producing a chain-shaped unsaturated carboxylic acid compound or a geometric isomer thereof, represented by the formula (2), wherein the compound or a geometric isomer thereof is used to produce the chain-shaped unsaturated carboxylic acid compound or a geometric isomer thereof, represented by the formula (3) 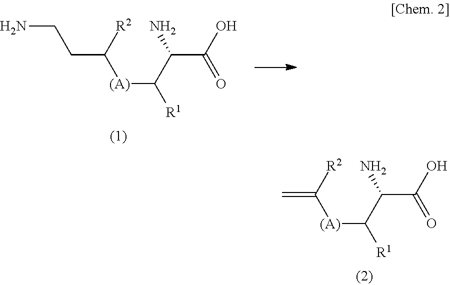
[in the formulas, (A) represents a linear hydrocarbon group having 0 to 5 carbon atoms, which may be substituted, and when the number of carbon atoms is 2 to 5, it may form a double bond between adjacent carbon atoms. R¹ and R² each independently represent a hydrogen atom, a linear or branched alkyl group having 1 to 5 carbon atoms, a linear or branched alkoxy group having 1 to 5 carbon atoms, or a hydroxy group.].

3. A method for producing a chain-shaped unsaturated hydrocarbon compound or a geometric isomer thereof, represented by the following formula (4), further comprising the steps of: producing the chain-shaped unsaturated carboxylic acid compound or a geometric isomer thereof, represented by the formula (3) by the method according to claim 1, and decarboxylating a carboxyl group from the unsaturated carboxylic acid compound or a geometric isomer thereof, in the presence of ferulic acid decarboxylase 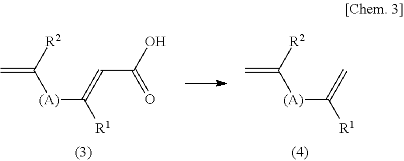
[in the formulas, (A) represents a linear hydrocarbon group having 0 to 5 carbon atoms, which may be substituted, and when the number of carbon atoms is 2 to 5, it may form a double bond between adjacent carbon atoms. R¹ and R² each independently represent a hydrogen atom, a linear or branched alkyl group having 1 to 5 carbon atoms, a linear or branched alkoxy group having 1 to 5 carbon atoms, or a hydroxy group.].

4. The method according to claim 1, where the phenylalanine ammonia lyase has at least one of the following features (1) to (5):
(1) position 108 in the amino acid sequence set forth in SEQ ID NO: 2, or an amino acid corresponding to the position, is methionine, phenylalanine, or valine,
(2) position 107 in the amino acid sequence set forth in SEQ ID NO: 2, or an amino acid corresponding to the position, is tryptophan,
(3) position 219 in the amino acid sequence set forth in SEQ ID NO: 2, or an amino acid corresponding to the position, is isoleucine,
(4) position 223 in the amino acid sequence set forth in SEQ ID NO: 2, or an amino acid corresponding to the position, is isoleucine,
(5) position 104 in the amino acid sequence set forth in SEQ ID NO: 2, or an amino acid corresponding to the position, is alanine.

5. A phenylalanine ammonia lyase variant into which at least one of the following amino acid substitutions (1) to (5) has been introduced:
(1) position 108 in the amino acid sequence set forth in SEQ ID NO: 2, or an amino acid corresponding to the position, is substituted with methionine, phenylalanine, or valine,
(2) position 107 in the amino acid sequence set forth in SEQ ID NO: 2, or an amino acid corresponding to the position, is substituted with tryptophan,
(3) position 219 in the amino acid sequence set forth in SEQ ID NO: 2, or an amino acid corresponding to the position, is substituted with isoleucine,
(4) position 223 in the amino acid sequence set forth in SEQ ID NO: 2, or an amino acid corresponding to the position, is substituted with isoleucine,
(5) position 104 in the amino acid sequence set forth in SEQ ID NO: 2, or an amino acid corresponding to the position, is substituted with alanine.

6. A DNA encoding the phenylalanine ammonia lyase variant according to claim 5.

7. A vector comprising the DNA according to claim 6.

8. A host cell into which the DNA according to claim 6 or the vector comprising the DNA according to claim 6 has been introduced.

9. A method for producing a phenylalanine ammonia lyase variant, comprising: culturing the host cell according to claim 8 and collecting a protein expressed in the host cell.

10. A method for producing a phenylalanine ammonia lyase variant, comprising the step of: introducing at least one of the following amino acid substitutions (1) to (5) into a phenylalanine ammonia lyase:
(1) substituting position 108 in the amino acid sequence set forth in SEQ ID NO: 2, or an amino acid corresponding to the position, with methionine, phenylalanine, or valine,
(2) substituting position 107 in the amino acid sequence set forth in SEQ ID NO: 2, or an amino acid corresponding to the position, with tryptophan,
(3) substituting position 219 in the amino acid sequence set forth in SEQ ID NO: 2, or an amino acid corresponding to the position, with isoleucine,
(4) substituting position 223 in the amino acid sequence set forth in SEQ ID NO: 2, or an amino acid corresponding to the position, with isoleucine,
(5) substituting position 104 in the amino acid sequence set forth in SEQ ID NO: 2, or an amino acid corresponding to the position, with alanine.
